(19) United States
(12) Patent Application Publication
(10) Pub. No.: US 2024/0071617 A1
(43) Pub. Date: Feb. 29, 2024

(54) ELECTRONIC DEVICE

(71) Applicant: KYOCERA Corporation, Kyoto-shi, Kyoto (JP)

(72) Inventor: Hiromi AJIMA, Kawasaki-shi, Kanagawa (JP)

(21) Appl. No.: 18/272,059

(22) PCT Filed: Jan. 12, 2022
(86) PCT No.: PCT/JP2022/000777

§ 371 Date: Jul. 12, 2023

(30) Foreign Application Priority Data
Jan. 15, 2021 (JP) 2021-005250

(51) Int. Cl. G16H 50/20; A61B 5/00; A61B 5/021

(52) U.S. Cl. CPC G16H 50/20; A61B 5/02116; A61B 5/7235

(57) ABSTRACT

An electronic device for generating a learning model to be used for estimation of a blood pressure level of a subject generates a learning model indicating a relationship between a blood pressure level and a pulse wave associated with the blood pressure level, based on an index of a pulse wave of a person at a first time point and an index of a pulse wave of the person at a second time point later than the first time point.

### CROSS-REFERENCE TO RELATED APPLICATIONS

The present application claims priority to Japanese Patent Application No. 2021-5250 filed in Japan on Jan. 15, 2021, the entire disclosure of which is incorporated herein by reference.

### TECHNICAL FIELD

The present disclosure relates to an electronic device, a method for controlling the electronic device, and a program.

### BACKGROUND OF INVENTION

A known electronic device measures biological information from a target region such as a wrist of a subject. More specifically, a proposed electronic device measures or estimates a blood pressure or the like of a subject from a pulse wave detected at a target region such as a wrist of the subject. For example, Patent Literature 1 discloses a sphygmomanometer that measures a change in blood pressure from a pulse wave of a subject.

### CITATION LIST

### Patent Literature

Patent Literature 1: Japanese Unexamined Patent Application Publication No. 2016-?119

### SUMMARY

An electronic device according to an embodiment is
an electronic device for generating a learning model to be used for estimation of a blood pressure level of a subject.

The electronic device generates a learning model indicating a relationship between a blood pressure level and a pulse wave associated with the blood pressure level, based on an index of a pulse wave of a person at a first time point and an index of a pulse wave of the person at a second time point later than the first time point.

An electronic device according to an embodiment estimates a blood pressure level of a subject by
using a learning model generated based on an index of a pulse wave of a person at a first time point and an index of a pulse wave of the person at a second time point later than the first time point, the learning model indicating a relationship between a blood pressure level and a pulse wave associated with the blood pressure level,
estimation of the blood pressure level of the subject being based on an index of a pulse wave acquired by a sensor, the index including an index of a pulse wave of the subject before a meal and an index of a pulse wave of the subject at another timing.

A method for control ling an electronic device according to an embodiment includes:
using a learning model generated based on an index of a pulse wave of a person at a first time point and an index of a pulse wave of the person at a second time point later than the first time point, the learning model indicating a relationship between a blood pressure level and a pulse wave associated with the blood pressure level; and
estimating a blood pressure level of a subject, based on an index of a pulse wave acquired by a sensor, the index including an index of a pulse wave of the subject before a meal and an index of a pulse wave of the subject at another timing.

A program according to an embodiment may is
a program for causing an electronic device to perform:
using a learning model generated based on an index of a pulse wave of a person at a first time point and an index of a pulse wave of the person at a second time point later than the first time point, the learning model indicating a relationship between a blood pressure level and a pulse wave associated with the blood pressure level; and
estimating a blood pressure level of a subject, based on an index of a pulse wave acquired by a sensor, the index including an index of a pulse wave of the subject before a meal and an index of a pulse wave of the subject at another timing.

### BRIEF DESCRIPTION OF THE DRAWINGS

FIG. 1 is a diagram illustrating a manner of using an electronic device according to an embodiment.

FIG. 2 is a diagram describing a target region of a subject.

FIG. 3 is a diagram illustrating the appearance of an electronic device according to an embodiment.

FIG. 4 is a diagram illustrating the appearance of an electronic device according to an embodiment.

FIG. 5 is a diagram illustrating the appearance of an electronic device according to an embodiment.

FIG. 6 is a diagram illustrating the appearance of an electronic device according to an embodiment.

FIG. 7 is a diagram illustrating an electronic device according to an embodiment and a wrist of the subject.

FIG. 8 is a diagram illustrating a cross section of an electronic device according to an embodiment.

FIG. 9 is a diagram illustrating the cross section of an electronic device according to an embodiment.

FIG. 10 is a diagram illustrating a manner of using an electronic device according to an embodiment.

FIG. 11 is a functional block diagram illustrating a schematic configuration of an electronic device according to an embodiment.

FIG. 12 is a diagram illustrating an example of a pulse wave acquired with a sensor unit.

FIG. 13 is a diagram illustrating a time variation in calculated AI.

FIG. 14 is a diagram illustrating a calculated AI and a measurement result of blood glucose level.

FIG. 15 is a diagram illustrating the relationship between the calculated AI and the blood glucose level.

FIG. 16 is a diagram illustrating an example of a pulse wave acquired with a sensor unit.

FIG. 17 is a diagram illustrating an example of results of blood pressure level estimation.

FIG. 18 is a diagram illustrating an example of results of blood pressure level estimation.

FIG. 19 is a diagram illustrating an example of results of blood pressure level estimation.

FIG. 20 is a diagram illustrating an example of results of blood glucose level estimation.

FIG. 21 is a flowchart illustrating the operation of an electronic device according to an embodiment.

FIG. 22 is a flowchart illustrating the operation of an electronic device according to an embodiment.

FIG. 23 is a schematic diagram illustrating a schematic configuration of a system according to an embodiment.

### DESCRIPTION OF EMBODIMENTS

High-accuracy estimation of blood pressure from a pulse wave of a subject is useful. The present disclosure provides an electronic device, a method for controlling the electronic device, and a program that enable high-accuracy estimation of a blood pressure of a subject. The present disclosure can provide an electronic device, a method for controlling the electronic device, and a program that enable high-accuracy estimation of a blood pressure of a subject. An embodiment will be described in detail hereinafter with reference to the drawings.

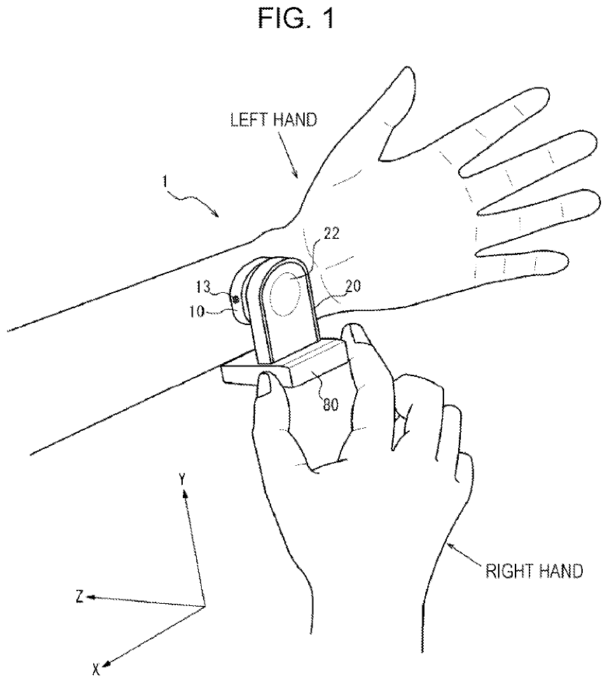

FIG. 1 is a diagram describing a manner of using an electronic device according to an embodiment. That is, FIG. 1 is a diagram illustrating how a subject measures biological information by using an electronic device according to an embodiment.

As illustrated in FIG. 1, an electronic device 1 according to an embodiment is capable of measuring biological information of a subject from, for example, a portion of the subject, such as a wrist of the subject, as a target region. In the example illustrated in FIG. 1, the electronic device 1 is in abutment against a target region of the left wrist of the subject. In the example illustrated in FIG. 1, the electronic device 1 is in abutment against a target region, and the target region is a wrist portion present on the way from the palm toward the elbow of the left hand of the subject. As described below, for example, the electronic device 1 can be free-standing on a horizontal surface such as on a table or a desk when not in abutment against the target region of the subject, such as before measurement.

As illustrated in FIG. 1, the electronic device 1 according to an embodiment includes a housing 10, a support 20, and a base 80. The housing 10 may include a switch 13 that turns on/off the power supply of the electronic device 1. As described below, the housing 10 includes a sensor 50 capable of detecting pulsation in the target region of the subject. The support 20 includes a rear surface portion 22. The rear surface portion 22 may be pressed by the subject or the like. As described below, the support 20 may include an extension portion 24 that is extendable. The base 80 supports the support 20 in an upright state. The functional units of the electronic device 1 will further be described below.

The positive direction of the Y axis illustrated in FIG. 1 is also referred to as an “upward” direction, if necessary. The negative direction of the Y axis illustrated in FIG. 1 is also referred to as a “downward” direction, if necessary. That is, the upward direction and the downward direction illustrated in FIG. 1 may be substantially the same as the upward direction and the downward direction when viewed from the viewpoint of the subject, respectively.

In FIG. 1, a portion of the electronic device 1 viewed from a viewpoint directed to the positive direction of the Z axis is referred to as a “rear surface” of the electronic device 1. That is, in FIG. 1, the rear surface of the electronic device 1 is a portion of the support 20 of the electronic device 1 where the rear surface portion 22 is viewed in plan. In FIG. 1, a portion of the electronic device 1 viewed from a viewpoint directed to the negative direction of the Z axis is referred to as a “front surface” of the electronic device 1. That is, in FIG. 1, that is, in FIG. 1, the front surface of the electronic device 1 is a portion of the housing 10 of the electronic device 1 where a surface to be brought into abutment against the target region of the subject is viewed in plan.

Before the measurement of biological information of the subject by using the electronic device 1 illustrated in FIG. 1, the following preparation may be carried out, for example. First, the subject may place the arm to be subjected to measurement of the biological information (in the example illustrated in FIG. 1, the left arm of the subject) on, for example, a stable stand or the like such as a table or a desk. In FIG. 1, the stand described above, such as a table or a desk, may have a deck (top plate) parallel to the XZ plane (i.e., perpendicular to the Y axis) illustrated in the drawing, for example. That is, the subject may place the arm to be subjected to measurement of the biological information on a stand or the like having a top plate perpendicular to the Y axis illustrated in the drawing. At this time, the palm of the hand to be subjected to measurement of the biological information of the subject (the left hand illustrated in FIG. 1) may be directed toward the negative direction of the Z axis illustrated in the drawing or directed toward a direction slightly shifted to the positive direction of the Y axis from the negative direction of the Z axis.

Then, the subject may place the electronic device 1 on, for example, a stable stand or the like such as a table or a desk so that the electronic device 1 can be free-standing. The electronic device 1 may be placed in a free-standing manner such that, for example, the bottom surface of the base 80 abuts against the deck (top plate) of the stand described above such as a table or a desk. At this time, the subject may bring the housing 10 of the electronic device 1 into abutment against the target region so that the sensor 50 of the electronic device 1 is located at a position where pulsation in the target region can be satisfactorily detected. Alternatively, the subject may bring the target region into abutment against the housing 10 of the electronic device 1. In this case, the subject may position the electronic device 1 by using the hand not to be subjected to measurement of the biological information (in the example illustrated in FIG. 1, the right hand of the subject).

Then, as illustrated in FIG. 1, the subject may press the base 80 of the electronic device 1 against the deck (top plate) of the stand, such as a table or a desk, by using a finger or the like of the hand not to be subjected to measurement of the biological information (in the example illustrated in FIG. 1, the right hand of the subject). As a result, the position of the electronic device 1 is secured on the table or desk. In the example illustrated in FIG. 1, the base 80 of the electronic device 1 is pressed against the table or desk by the subject with the thumb and index finger of the right hand. The base 80 stands upright, with the support 20 secured. Accordingly, as illustrated in FIG. 1, the electronic device 1 according to an embodiment measures biological information of the subject while being pressed against the target region. The fingers with which the subject presses the base 80 against the table or desk are not limited to the thumb and index finger of the right hand. The subject may press the base 80 against the table or desk with a finger other than the thumb and index finger of the right hand. Further, the subject may not necessarily press the base 80 of the electronic device 1 against the table or desk, but may press, for example, the support 20 or the like against the table or desk. The base 80 or the support 20 of the electronic device 1 may be pressed in any manner if it is pressed against the table or desk with an appropriate pressing force. The base 80 or the support 20 of the electronic device 1 may be placed on a table, a desk, or any other stand made of wood, iron, plastic, glass, rubber, resin, or any other material, and any combination thereof.

The electronic device 1 can detect pulsation in the target region upon being brought into abutment against the target region of the subject. The target region of the subject may be, for example, a region of the body where the ulnar artery or radial artery of the subject is present beneath the skin. The target region of the subject is not limited to a region of the body where the ulnar artery or radial artery of the subject is present beneath the skin, and may be any region of the body where the pulsation of the subject is detectable. FIG. 1 illustrates the electronic device 1 in abutment against a target region that is a region of the body where the radial artery is located beneath the skin of the wrist of the subject.

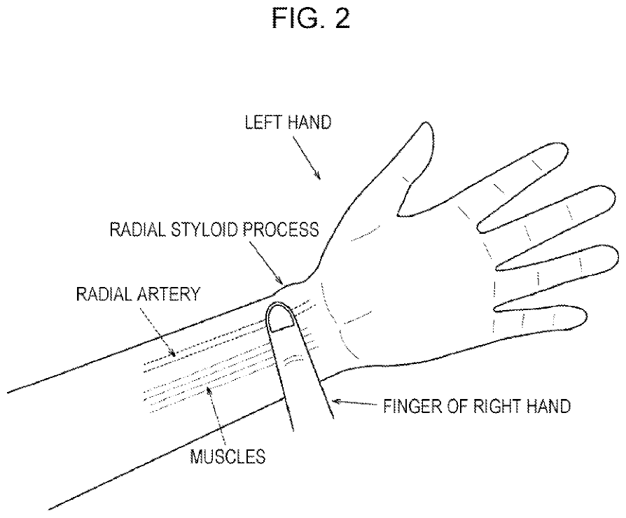

FIG. 2 is a diagram describing the target region of the subject. More specifically, FIG. 2 illustrates an example in which the subject searches their target region for a portion where pulsation is satisfactorily detectable before measuring biological information by using the electronic device 1. That is, FIG. 2 illustrates how the subject searches the target region of their left hand for a portion where pulsation is satisfactorily detectable, by using a finger of their right hand. In FIG. 2, as in FIG. 1, it ay be assumed that the subject has placed their left arm on a stand or the like such as a table or a desk. In FIG. 2, the radial artery and the muscles beneath the skin of the arm of the subject are indicated by broken lines, chain lines, or the like.

As described above, the subject may bring the housing 10 of the electronic device 1 into abutment against the target region such that the sensor 50 of the electronic device 1 is located at a position where pulsation is satisfactorily detectable. The position on the target region of the subject where pulsation is satisfactorily detectable differs depending on the subject (individual difference). Accordingly, the subject may search their target region for a position where pulsation is satisfactorily detectable before measuring biological information by using the electronic device 1.

In many cases, the position where pulsation is satisfactorily detectable near the wrist of the subject is a position where the radial artery runs beneath the skin and where the radial styloid process is present beneath the skin, or near this position. In a portion where the radial artery runs above the radial styloid process, the radial artery is located above the radial styloid process, which is relatively stiff. At this position, the movement of the radial artery that contracts due to pulsation is more easily transmitted toward the skin of the subject, which is relatively soft, than toward the radial styloid process, which is relatively stiff. Accordingly, the position described above may be set as the target region for the measurement of biological information of the subject by using the electronic device 1 according to an embodiment.

As illustrated in FIG. 2, it is assumed that the subject has found a good pulse at, for example, a position illustrated in the drawing around the wrist of their left hand by using a fingertip of their right hand. In this case, the subject may set the position at which the subject has found a good pulse by using the fingertip of their right hand as the target region. In this way, as illustrated in FIG. 1, the subject may bring the housing 10 of the electronic device 1 into abutment against the target region. Setting the target region such that the target region includes the positions of many muscles illustrated in FIG. 2 may make it difficult to satisfactorily transmit the pulsation of the radial artery to the housing 10 (and the sensor 50) of the electronic device 1. Accordingly, when bringing the housing 10 of the electronic device 1 into abutment against the target region, the subject may arrange the electronic device 1 so that the housing 10 (and the sensor 50) of the electronic device 1 can be pressed against the radial artery while avoiding the muscles as much as possible. A portion of the housing 10 of the electronic device 1 to be brought into abutment against the target region of the subject will further be described below. Further, as illustrated in FIG. 1, when measuring biological information of the subject by using the electronic device 1, the subject may be in a psychological condition such that the entire body is relaxed, and the palm of the hand to be subjected to measurement of the biological information (for example, the left hand) may be slightly opened.

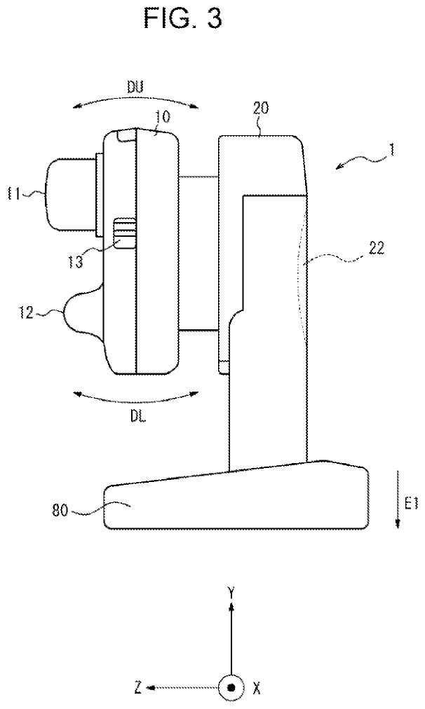

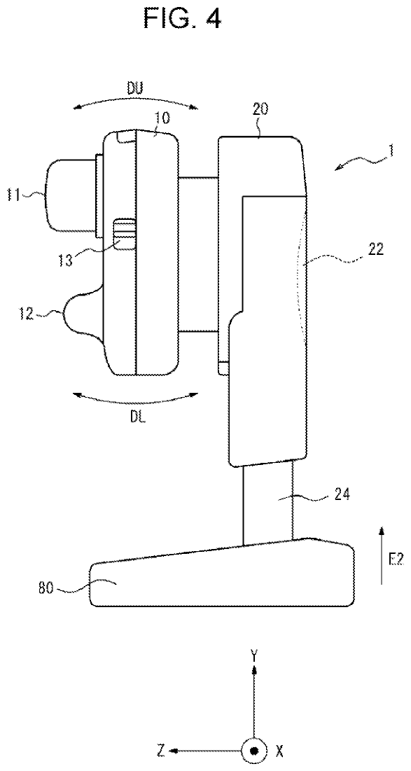

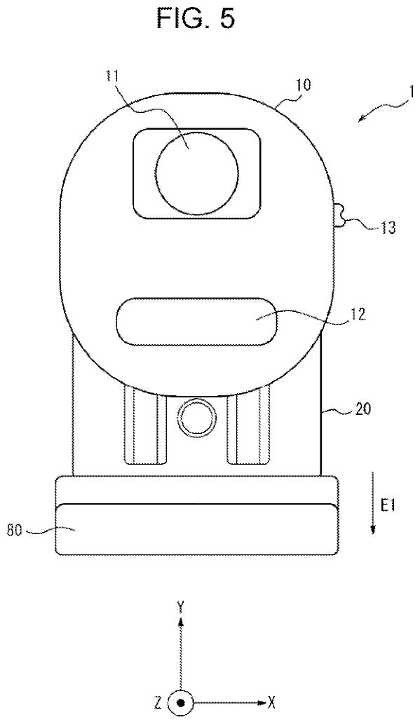

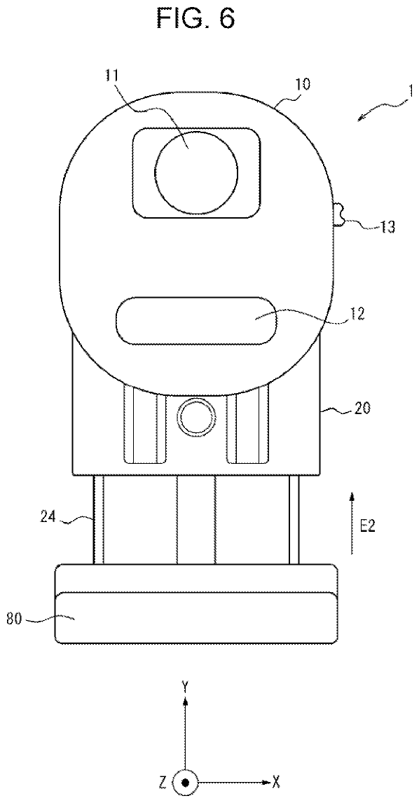

The configuration of the electronic device 1 according to an embodiment will further be described. FIGS. 3 and 4 are diagrams illustrating the electronic device 1 illustrated in FIG. 1 when viewed from a viewpoint directed to the negative direction of the X axis. That is, FIGS. 3 and 4 are diagrams illustrating the right side surface of the electronic device 1 illustrated in FIG. 1. FIGS. 5 and 6 are diagrams illustrating the electronic device 1 illustrated in FIG. 1 when viewed from a viewpoint directed to the negative direction of the Z axis. That is, FIGS. 5 and 6 are diagrams illustrating the front surface of the electronic device 1 illustrated in FIG. 1.

As illustrated in FIGS. 3 to 6, the electronic device 1 includes the housing 10, the support 20, and the base 80. In the electronic device 1, as described bellow, the housing 10 and the support 20 are connected through an elastic member. As illustrated in FIGS. 3 to 6, the support 20 supports the housing 10 on a side of the support 20. That is, the housing 10 is supported on a side of the support 20. The base 80 allows the support 20 to stand upright. The housing 10, the support 20, and/or the base 80 may be made of, for example, a material such as ceramic, iron, any other metal, resin, plastic, or aluminum. The housing 10, the support 20, and/or the base 80 may be made of a hard and lightweight material. The material of the housing 10, the support 20, and/or the base 80 is not limited, and may have strength enough to function as a measurement device. Further, the material of the housing 10, the support 20, and/or the base 80 is not excessively heavy and may be relatively light.

The sizes of the housing 10, the support 20, and the base 80 of the electronic device 1 are not limited, and may be relatively small in terms of portability, ease of measurement, and/or the like. For example, the electronic device 1 may have a size such that, for example, the entire electronic device 1 is included in a cube or a rectangular parallelepiped having sides of about 7 cm each. However, in one embodiment, the size of the entire electronic device 1 may be larger or smaller than the size described above. Further, the shapes of the individual portions of the electronic device 1, such as the housing 10, the support 20, and the base 80, are not limited to the illustrated shapes, and various shapes may be used in teems of functionality of a measurement device, design viewpoint, and/or the like. In particular, the base 80 allows the support 20 to stand upright. Accordingly, the base 80 may be shaped to have a bottom area such that the electronic device 1 including the housing 10 and the support 20 can stand upright. Alternatively, the base 80 may have a bottom area such that the electronic device 1 can be free-standing on a horizontal surface.

As described below, the housing 10 and the support 20 can move freely to some extent with respect to each other. That is, in the electronic device 1, the support 20 can move freely to some extent even while the housing 10 is secured. In the electronic device 1, the housing 10 can move freely to some extent even while the support 20 is secured. For example, as illustrated in FIGS. 3 and 4, in the electronic device 1, the housing 10 can move freely to some extent in a direction indicated by an arrow DU and/or a direction indicated by arrow DL illustrated in the drawings.

As illustrated in FIGS. 3 to 6, the support 20 of the electronic device 1 may include, for example, the extension portion 24 therein. The extension portion 24 is extendable from the support 20. FIGS. 3 and 5 illustrate a state in which the extension portion 24 is not extended from the support 20. In contrast, FIGS. 4 and 6 illustrate a state in which the extension portion 24 is extended from the support 20. That is, in FIGS. 3 and 5, the extension portion 24 is extended in a direction indicated by an arrow E1, thus making it possible to extend the extension portion 24 such that, as illustrated in FIGS. 4 and 6, the extension portion 24 projects from the support 20. In contrast, in FIGS. 4 and 6, the extension portion 24 is contracted in a direction indicated by an arrow E2, thus making it possible to return the extension portion 24 to the original position, as illustrated in FIGS. 3 and 5. In the electronic device 1 according to an embodiment, therefore, the extension portion 24 may be extended or contracted to make the length of the support 20 in the upward/downward direction adjustable.

In addition, the length of the support 20 in the upward/downward direction can be adjusted by the extension portion 24 to make the position of the housing 10 in the upward/downward direction (height direction) adjustable. Accordingly, even if the thickness of the left wrist of the subject illustrated in FIG. 1 differs to some extent from individual to individual, the position at which the housing 10 is brought into abutment against the target region of the subject can be adjusted in accordance with the position of the target region of the subject in the upward/downward direction. In this manner, in the electronic device 1 according to an embodiment, the support 20 may be extendable or contractible in a predetermined direction, such as the direction indicated by the arrow E1 and/or the arrow E2, to make the position of the housing 10 in the height direction adjustable.

The extension portion 24 may be extendable steplessly from the support 20. That is, the extension portion 24 may be configured such that the extension portion 24 can be positioned at any position up to a predetermined length, for example. With this configuration, even if the thickness of the wrist of the subject, including the target region, differs from individual to individual, the position at which the housing 10 of the electronic device 1 is brought into abutment against the target region of the subject can be finely adjusted.

The extension portion 24 may be extendable stepwise from the support 20. That is, the extension portion 24 may include, for example, a mechanism that facilitates positioning at a plurality of predetermined positions up to a predetermined length. For example, the extension portion 24 may include a mechanism such as a multi-stage stay that is locked in multiple stages when the extension portion 24 is extended or contracted from the support 20. With this configuration, when the subject measures biological information by using the electronic device 1, for example, the same measurement environment as that of the previous measurement is easily reproduced. In this manner, in the electronic device 1 according to an embodiment, for example, the support 20 may include the extension portion 24 and may be extendable or contractible stepwise in a predetermined direction, such as the direction indicated by the arrow E1 and/or the arrow E2.

As illustrated in FIGS. 3 to 6, the housing 10 of the electronic device 1 may include a first abutment portion 11 as a portion to be brought into abutment against the target region of the subject. The first abutment portion 11 may be disposed on the side of the housing 10 closer to the target region. The first abutment portion 11 may function as a member such as a pulse contact portion, for example. As illustrated in FIGS. 3 to 6, the housing 10 of the electronic device 1 may further include a second abutment portion 12 as a portion to be brought into abutment against the target region of the subject or the vicinity of the target region. The second abutment portion 12 may be brought into abutment against the vicinity of the position against which the first abutment portion 11 abuts in the target region of the subject. The second abutment portion 12 may also be disposed on the side of the housing 10 closer to the target region (side closer to the wrist of the subject).

As described above, the first abutment portion 11 is a member to be appropriately brought into abutment against the target region of the subject when the electronic device 1 measures biological information of the subject. Accordingly, the first abutment portion 11 may have a size such that, for example, the first abutment portion 11 is appropriately brought into abutment against a region of the body where the ulnar artery or radial artery of the subject is present beneath the skin. For example, as illustrated in FIGS. 5 and 6, the first abutment portion 11 may have a width of about 1 cm to 1.5 cm in the X-axis direction or the Y-axis direction. Alternatively, the first abutment portion 11 may have a width other than about 1 cm to 1.5 cm in the X-axis direction or the Y-axis direction.

The first abutment portion 11 and the second abutment portion 12 may be made of, for example, a material such as ceramic, iron, any other metal, resin, plastic, or aluminum. The first abutment portion 11 and the second abutment portion 12 may be made of a hard and lightweight material. The material of the first abutment portion 11 and the second abutment portion 12 is not limited. The material of the first abutment portion 11 and the second abutment portion 12 may have strength enough to function as a measurement device and may be relatively lightweight, like the housing 10 and/or the support 20.

As illustrated in FIGS. 3 to 6, the housing 10 of the electronic device 1 may include the switch 13. The switch 13 may be, for example, a switch that switches on/off of the power supply of the electronic device 1. The switch 13 may be, for example, a switch that causes the electronic device 1 to start measurement of biological information. In the example illustrated in FIGS. 3 to 6, the switch 13 is a slide switch. However, the switch 13 may be any switch such as a push button switch, for example. For example, the switch 13 is a push button switch. In this case, various operations of the electronic device 1 may be supported in accordance with the number of times the switch 13 is pressed, the amount of time during which the switch 13 is pressed, and/or the like. The location where the switch 13 is arranged is not limited to that in the example illustrated in FIGS. 3 to 6, and the switch 13 may be arranged in any location. For example, the switch 13 may be arranged in the support 20.

Measurement of biological information by using the electronic device 1 according to an embodiment will be described.

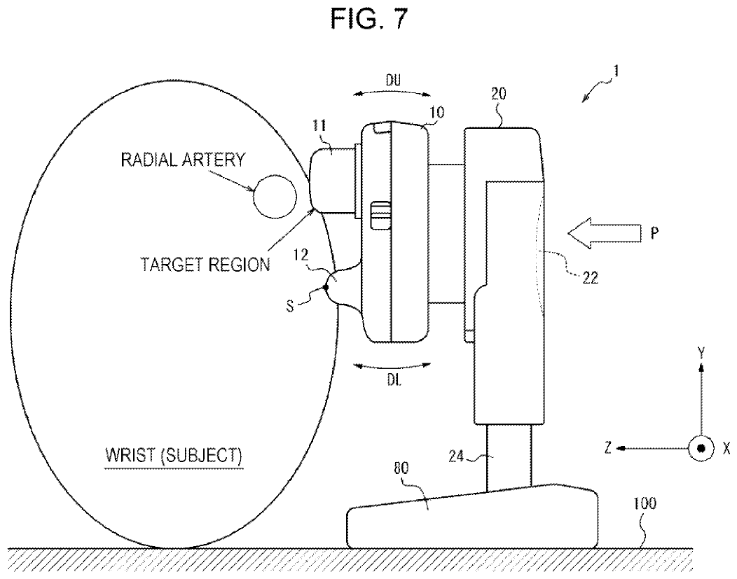

FIG. 7 illustrates how the subject measures biological information by using the electronic device 1. FIG. 7 is a diagram illustrating the electronic device 1 illustrated in FIG. 1 when viewed from a side, together with a cross section of the wrist of the subject. That is, FIG. 7 is a diagram illustrating the electronic device 1 illustrated in FIG. 1 when viewed from a viewpoint directed to the negative direction of the X axis, together with a cross section of the wrist of the subject.

As illustrated in FIG. 7, the left wrist of the subject is placed on an upper surface of a deck (top plate) 100 of a stand such as a table or a desk. The deck (top plate) 100 of the stand, such as a table or a desk, is also referred to simply as a “horizontal surface 100”. The horizontal surface 100 may be a surface that is horizontal, and may include not only a surface that is exactly horizontal but also a surface that is substantially horizontal. As illustrated in FIG. 7, furthermore, the electronic device 1 is free-standing on the horizontal surface 100 such that a lower end, that is, a bottom surface, of the base 80 that allows the support 20 to stand upright comes into contact with the horizontal surface 100. That is, in the electronic device 1 according to an embodiment, the base 80 may allow the support 20 to stand upright. Further, the base 80 may allow the support 20 to stand upright such that the electronic device 1 is free-standing on the horizontal surface 100. In the example illustrated in FIG. 7, the extension portion 24 is somewhat extended from the support 20 of the electronic device 1. For example, the electronic device 1 can start measurement of biological information, with the base 80 (or the support 20) pressed against the horizontal surface 100 by the subject with the right hand or the like. Alternatively, the electronic device 1 may be used without the bottom surface of the base 80 coming into contact with the upper surface of the horizontal surface 100 (i.e., with the base 80 suspended from the horizontal surface 100). In this case, for example, the electronic device 1 may start measurement of biological information upon being pressed by the subject in a direction indicated by an arrow P illustrated in FIG. 7 with the right hand or the like.

As illustrated in FIG. 7, the first abutment portion 11 may be brought into direct or indirect contact with the target region of the subject. As illustrated in FIG. 7, the second abutment portion 12 may be brought into direct or indirect contact with the vicinity of the region of the body where the first abutment portion 11 comes into contact with the target region of the subject. As illustrated in FIG. 7, a surface including the target region of the wrist of the subject typically has a curved shape. If the first abutment portion 11 and the second abutment portion 12 of the housing 10 have the same length in the Z-axis direction, the first abutment portion 11 may be away from (the target region of) the wrist of the subject while the second abutment portion 12 is in contact with the wrist of the subject. In one embodiment, accordingly, as illustrated in FIG. 7, the length of the first abutment portion 11 in the Z-axis direction may be longer than the length of the second abutment portion in the Z-axis direction. With this shape, the first abutment portion 11 can be appropriately brought into abutment against the target region of the subject while the second abutment portion 12 comes into contact with a portion of the wrist of the subject (for example, a portion S illustrated in FIG. 7).

In this manner, in one embodiment, the first abutment portion 11 may protrude from the housing 10 more than the second abutment portion 12 in the Z-axis direction illustrated in FIG. 7, for example. That is, the length by which the first abutment portion 11 protrudes from the housing 10 in the Z-axis positive direction may be larger than the length by which the second abutment portion 12 protrudes from the housing 10 in the Z-axis positive direction. The shape of the first abutment portion 11 is not limited to the shape illustrated in FIGS. 3 to 7, and may be any shape that enables the first abutment portion 11 to appropriately abut against the target region of the subject. Likewise, the shape of the second abutment portion 12 is not limited to the shape illustrated in FIGS. 3 to 7, and may be any shape that enables the second abutment portion 12 to appropriately abut against a portion of the wrist of the subject (for example, the portion S illustrated in FIG. 7).

As illustrated in FIG. 7, the support 20 of the electronic device 1 may include the rear surface portion 22. The rear surface portion 22 may be a portion of the electronic device 1 that is pressed by the subject with a fingertip or the like. That is, by pressing the rear surface portion 22 with a fingertip or the like, the subject or the like can measure biological information by using the electronic device 1 even if the base 80 or the support 20 is not pressed against the horizontal surface 100. As illustrated in FIG. 7, the rear surface portion 22 may be formed on the rear side of the support 20 (the surface directed to the Z-axis negative direction). In the example illustrated in FIG. 7, the rear surface portion 22 is formed in a location slightly above (in the Y-axis positive direction) the center of the support 20. However, the rear surface portion 22 may be formed in any location in accordance with the manner in which the electronic device 1 measures biological information, such that, for example, the rear surface portion 22 is formed substantially at the center of the support 20.

In the example illustrated in FIG. 7, furthermore, the rear surface portion 22 is illustrated as a shallow concave portion formed in the support 20. However, the shape of the rear surface portion 22 is not limited to a shallow concave portion. For example, the rear surface portion 22 may be formed as a shallow convex portion or the like formed in the support 20. Alternatively, the rear surface portion 22 may merely be, for example, a mark painted on the support 20 with paint or the like. In the electronic device 1, the rear surface portion 22 may be configured in any manner so long as it represents a portion to be pressed by the subject with a fingertip or the like.

In the electronic device 1 the first abutment portion 11 is brought into abutment against the target region such as the wrist of the subject, and the base 80 or the support 20 is pressed against the horizontal surface 100 by the subject with a fingertip or the like. As a result, the electronic device 1 is brought into the state illustrated in FIG. 1 or FIG. 7 for measurement of biological information. When the electronic device 1 is to be brought into abutment against the target region such as the wrist of the subject, the electronic device 1 may be positioned such that the first abutment portion 11 comes into abutment against the target region of the subject. At this time, as illustrated in FIG. 7, the electronic device 1 may be positioned such that, for example, the first abutment portion 11 comes into abutment against a region of the body where the ulnar artery or radial artery of the subject is present beneath the skin. That is, the target region for which the electronic device 1 according to an embodiment measures biological information of the subject may be, for example, a position where the radial artery or ulnar artery of the subject flows beneath the skin.

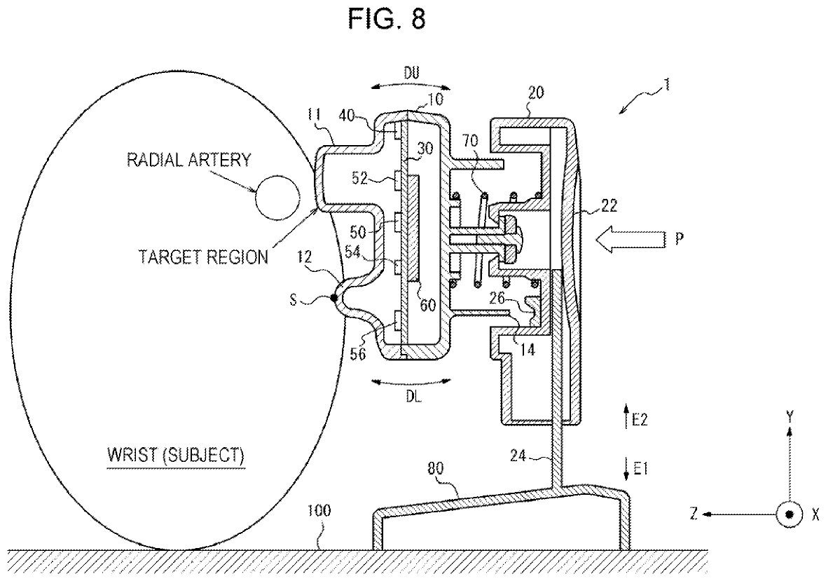

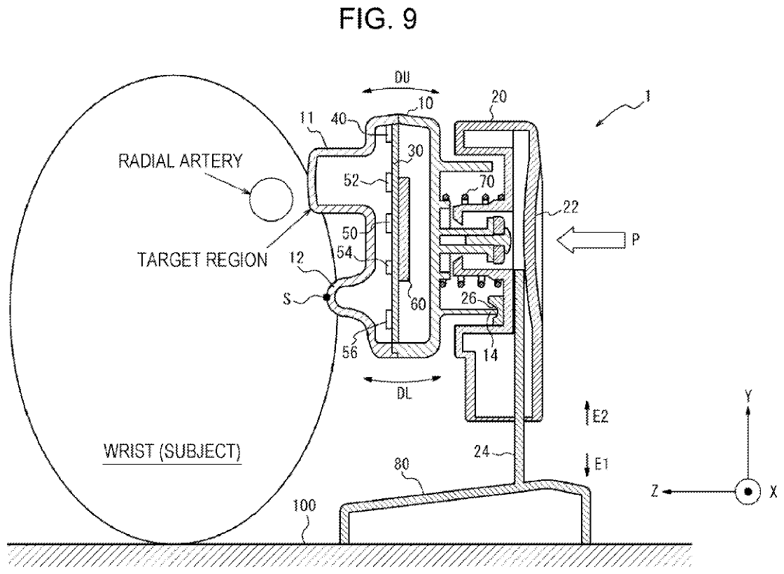

FIGS. 8 and 9 are diagrams illustrating a cross section of the electronic device 1, together with a cross section of the wrist of the subject. FIG. 8 is a diagram illustrating a cross section of the electronic device 1 illustrated in FIG. 7, together with a cross section of the wrist of the subject. FIG. 9 is a sectional view illustrating a state in which the base 80 is pressed against (secured to) the horizontal surface 100 to apply a force to the support 20 of the electronic device 1 illustrated in FIG. 8 in the direction indicated by the arrow P illustrated in the drawing. The force in the direction indicated by the arrow P may be the reaction of a force with which the target region of the subject presses (the housing 10 of) the electronic device 1.

As illustrated in FIGS. 8 and 9, the electronic device 1 includes, in appearance, the housing 10, the support 20, and the base 80. As described above, the housing 10 includes the first abutment portion 11 and the second abutment portion 12. The support 20 may include the rear surface portion 22 and the extension portion 24.

As illustrated in FIGS. 8 and 9, the housing 10 of the electronic device 1 may further include a substrate 30. The substrate 30 may be a typical circuit board on which various electronic components and the like can be arranged. In one embodiment, the substrate 30 may be built in the housing 10 of the electronic device 1.

Various electronic components may be arranged on the surfaces of the substrate 30 on the Z-axis negative and positive direction sides. In the example illustrated in FIGS. 8 and 9, a notification unit 40, the sensor 50, a control unit 52, a storage unit 54, and a communication unit 56 are arranged on the surfaces of the substrate 30 on the Z-axis negative and positive direction sides. The switch 13 described above may also be arranged on the substrate 30.

The notification unit 40 notifies the subject or the like of, for example, information such as a measurement result of biological information. The notification unit 40 may be, for example, a light-emitting unit such as a light-emitting diode (LED). Alternatively, the notification unit 40 may be a display device such as a liquid crystal display (LCD), an organic EL display (OELD: Organic Electro-Luminescence Display), or an inorganic EL display (IELD: Inorganic Electro-Luminescence Display). Such a display device employed as the notification unit 40 can display, for example, relatively detailed information such as the state of glucose metabolism or lipid metabolism of the subject.

The notification unit 40 may notify the subject of not only information such as a measurement result of biological information but also, for example, information such as on/off of the power supply of the electronic device 1 or whether biological information is being measured. At this time, for example, the notification unit 40 may notify the subject of information such as on/off of the power supply of the electronic device 1 or whether biological information is being measured by a different type of light emission from that when notifying the subject of information such as a measurement result of biological information.

In one embodiment, the notification unit 40 may not necessarily be a light-emitting unit. For example, the notification unit 40 may be a sound output unit such as a speaker or a buzzer. In this case, the notification unit 40 may notify the subject or the like of, for example, information such as a measurement result of biological information via various sounds, voices, or the like.

In one embodiment, the notification unit 40 may be, for example, a tactile sensation providing unit such as a vibrator or a piezoelectric element. In this case, the notification unit 40 may notify the subject or the like of, for example, information such as a measurement result of biological information via various types of vibration, tactile sensation feedback, or the like.

The sensor 50 includes, for example, an angular speed sensor and detects pulsation from the target region to acquire a pulse wave. The sensor 50 may detect a displacement of the first abutment portion 11 (pulse contact portion) based on the pulse wave of the subject. The sensor 50 may be, for example, an acceleration sensor or may be a sensor such as a gyro sensor. Alternatively, the sensor 50 may be an angular speed sensor. The sensor 50 will further be described below.

As illustrated in FIGS. 8 and 9, the sensor 50 is secured to the substrate 30. The substrate 30 is secured within the housing 10. The first abutment portion 11 is secured to the outside of the housing 10. Thus, a movement of the first abutment portion 11 is transmitted to the sensor 50 through the housing 10 and the substrate 30. Accordingly, the sensor 50 can detect the pulsation in the target region of the subject through the first abutment portion 11, the housing 10, and the substrate 30.

In the example illustrated in Ms. 8 and 9, the sensor 50 is arranged such that the sensor 50 is built in the housing 10. However, in one embodiment, the sensor 50 may not be entirely built in the housing 10. In one embodiment, the sensor 50 may be included in at least part of the housing 10. The sensor 50 may have any configuration in which a movement of at least one selected from the group consisting of the first abutment portion 11, the housing 10, and the substrate 30 is transmitted to the sensor 50.

The control unit 52 is a processor that controls and manages the entire electronic device 1, including the functional blocks of the electronic device 1. Further, the control unit 52 is a processor that calculates, from the acquired pulse wave, an index based on the propagation phenomenon of the pulse wave. The control unit 52 is a processor such as a CPU (Central Processing Unit) that executes a program specifying a control procedure and a program for calculating an index based on the propagation phenomenon of the pulse wave, and the programs are stored in a storage medium, such as the storage unit 54, for example. Further, the control unit 52 estimates a state related to glucose metabolism, lipid metabolism, or the like of the subject on the basis of the calculated index. The control unit 52 may send data to the notification unit 40.

The storage unit 54 stores programs and data. The storage unit 54 may include any non-transitory storage medium such as a semiconductor storage medium and a magnetic storage medium. The storage unit 54 may include a plurality of types of storage media. The storage unit 54 may include a combination of a portable storage medium, such as a memory card, an optical disk, or a magneto-optical disk, and a storage medium reading device. The storage unit 54 may include a storage device used as a temporary storage area such as a RAM (Random Access Memory). The storage unit 54 stores various types of information and/or programs for operating the electronic device 1, and also functions as a work memory. The storage unit 54 may store, for example, a measurement result of the pulse wave acquired by the sensor 50.

The communication unit 56 performs wired communication or wireless communication with an external device to transmit and receive various data. The communication unit 56 communicates with, for example, an external device that stores biological information of the subject to manage the health condition, and transmits the measurement result of the pulse wave measured by the electronic device 1 and/or the health condition estimated by the electronic device 1 to the external device. The communication unit 56 may be, for example, a communication module that supports Bluetooth (registered trademark), Wi-Fi, or the like.

As illustrated in FIGS. 8 and 9, a battery 60 may be arranged on the surface of the substrate 30 on the Z-axis negative direction side. In this case, a battery holder may be arranged on the surface of the substrate 30 on the Z-axis negative direction side to secure the battery 60 in position. The battery 60 may be any power supply, for example, a button battery (coin battery) such as CR2032. Alternatively, the battery 60 may be, for example, a rechargeable storage battery. The battery 60 may include, for example, a lithium-ion battery and a control circuit or the like for charging and discharging the lithium-ion battery, if necessary. The battery 60 may supply electric power to the functional units of the electronic device 1.

The arrangement of the notification unit 40, the sensor 50, the control unit 52, the storage unit 54, the communication unit 56, and the battery 60 is not limited to that in the example illustrated in FIGS. 8 and 9. For example, the functional units described above may be arranged at any positions on the substrate 30. The functional units described above may be each arranged on either side of the substrate 30, as necessary. The electronic device 1 may be connected to an external device in a wired or wireless manner. In this case, for example, at least some of the functional units such as the switch 13, the notification unit 40, the control unit 52, the storage unit 54, and the communication unit 56 may be included in the external device, as necessary.

As illustrated in FIGS. 8 and 9, in the electronic device 1, the end of the housing 10 on the Z-axis negative direction side is connected to the end of the support 20 on the Z-axis positive direction side. As illustrated in FIGS. 8 and 9, the housing 10 has, on the side thereof in the negative direction of the Z axis, a connection portion to be connected to the support 20. As illustrated in FIGS. 8 and 9, the support 20 has, on the side thereof in the positive direction of the Z axis, an opening into which the connection portion of the housing 10 is inserted. In the example illustrated in FIGS. 8 and 9, the connection portion of the housing 10 has a smaller size than the opening in the support 20 such that the connection portion of the housing 10 is insertable into the opening in the support 20. However, in one embodiment, the housing 10 may have an opening, and the support 20 may have an insertion portion. In this case, the opening in the housing 10 may have a larger size than the insertion portion of the support 20 such that the insertion portion of the support 20 is insertable into the opening in the housing 10 in both cases, the housing 10 and the support 20 may be movable freely to some extent without interfering with each other.

As illustrated in FIGS. 8 and 9, in the electronic device 1, the housing 10 and the support 20 are connected to each other through an elastic member 70. In the example illustrated in FIGS. 8 and 9, the housing 10 and the support 20 are directly connected by the elastic member 70. However, for example, the elastic member 70 may indirectly connect the housing 10 and the support 20. For example, in one embodiment, the elastic member 70 may connect any member of the housing 10 and any member of the support 20 to each other. The elastic member 70 may be an elastic member deformable along at least any one axis among three axes orthogonal to one another (for example, the Y axis, the Y axis, and the Z axis). The elastic member 70 is a three-dimensionally deformable member.

FIGS. 8 and 9 illustrate an example in which the elastic member 70 is a spring such as a compression coil spring. However, in one embodiment, the elastic member 70 may be, for example, any elastic body having appropriate elasticity, such as a spring, a resin, a sponge, or a silicone sheet, or may be any combination thereof. The elastic member 70 may be formed by, for example, a silicone sheet of a predetermined thickness having predetermined elasticity.

FIG. 8 illustrates a state in which no force (or a very weak force) is applied to the support 20 in the direction indicated by the arrow P. That is, FIG. 8 illustrates a state in which the support 20 is not (substantially) pressed toward the target region. In contrast, FIG. 9 illustrates a state in which a force is applied to the support 20 in the direction indicated by the arrow P. That is, FIG. 9 illustrates a state in which the support 20 is pressed toward the target region. Since such a pressing force deforms the elastic member 70, the length of the elastic member 70 illustrated in FIG. 9 in the Z-axis direction is shorter than the length of the elastic member 70 illustrated in FIG. 8 in the Z-axis direction. As described above, the force in the direction indicated by the arrow P illustrated in FIG. 8 or FIG. 9 may be a force generated in response to the base 80 or the support 20 being pressed against (secured to) the horizontal surface 100 by the subject or the like. Alternatively, the force in the direction indicated by the arrow P illustrated in FIG. 8 or FIG. 9 may be the reaction of a force with which the subject presses the target region against (the housing 10, the first abutment portion 11 or the second abutment portion 12, or the like of) the electronic device 1.

In the example illustrated in FIG. 8, the electronic device 1 includes a stopper mechanism that does not allow the housing 10 and the support 20 to be displaced a distance of a predetermined length or longer. That is, the electronic device 1 illustrated in FIG. 8 includes a mechanism that does not allow the housing 10 to be removed or fall off from the support 20 even while the electronic device 1 is not pressed in the direction indicated by the arrow P illustrated in the drawing. FIG. 8 illustrates a state in which the distance between the housing 10 and the support 20 is fixed, with the restoration force of the elastic member 70 maintained to some extent. In this situation, the housing 10 and the support 20 are not displaced a larger distance.

In the situation illustrated in FIG. 8, if a pressing force is applied in the direction indicated by the arrow P illustrated in the drawing, in contrast, as illustrated in FIG. 9, the elastic member 70 is deformed so as to contract. In the situation illustrated in FIG. 9, a protruding portion 14 of the housing 10 reaches and comes into contact with a receiving portion 26 of the support 20. If the pressing force in the direction indicated by the arrow P illustrated in the drawing becomes weaker than that in this state, a state can be implemented in which the protruding portion 14 of the housing 10 does not come into contact with the receiving portion 26 of the support 20 while the elastic member 70 is somewhat contracted. In this state, the housing 10 can be displaced freely to some extent with respect to the support 20 connected through the elastic member 70. Accordingly, the electronic device 1 can satisfactorily detect pulsation in the target region of the subject.

In FIGS. 8 and 9, the elastic member 70 is a spring such as a compression coil spring. However, as described above, for example, the elastic member 70 may be a silicone seed or the like of a predetermined thickness. In this case, the housing 10 and the support 20 may be bonded to the elastic member 70 with an adhesive, a double-sided adhesive tape, or the like. The elastic member 70 may be bonded to any other member such that the influence on the deformation of the elastic member 70 can be reduced. That is, the elastic member 70 may be appropriately deformable even when the elastic member 70 is bonded to any other member.

As described above, the electronic device 1 according to an embodiment includes the housing 10, the support 20, the sensor 50, the elastic member 70, and the base 80. The housing 10 includes, at least in part, the sensor 50. The sensor 50 is capable of detecting pulsation in a target region of a subject. The support 20 supports the housing 10 on a side of the support 20. The elastic member 70 is interposed between the housing 10 and the support 20. The base 80 allows the support 20 to stand upright. The base 80 may allow the support 20 to stand upright such that the electronic device 1 is free-standing on a horizontal surface.

As illustrated in FIGS. 8 and 9, in a state where the support 20 of the electronic device 1 is caused to stand upright by the base 80, the first abutment portion 11 of the housing 10 can come into contact with the target region of the subject, that is, the skin over the radial artery of the subject. As illustrated in FIGS. 8 and 9, the housing 10 is supported on a side of the support 20. When the base 80 or the support 20 is pressed against the horizontal surface 100 by the subject or the like, the positions of the base 80 and the support 20 on the horizontal surface 100 are fixed. In this state, in response to the subject pressing the target region against the housing 10 (alternatively, the first abutment portion 11 or the second abutment portion 12, or the like), the base 80 and the support 20 generate a reaction of the force toward the target region, that is, in the direction indicated by the arrow P. Due to the elastic force of the elastic body 140 arranged between the support 20 to which a force is applied in the direction indicated by the arrow P and the housing 10 including the sensor 50, the sensor 50 is urged toward the target region of the subject (together with the housing 10 and the first abutment portion 11). The first abutment portion 11, which is urged by the elastic force of the elastic member 70, comes into contact with the skin over the radial artery of the subject. In this case, the first abutment portion 11 is displaced in accordance with the movement of the radial artery of the subject, that is, the pulsation. Accordingly, the sensor 50, which operates in association with the first abutment portion 11, is also displaced in accordance with the movement of the radial artery of the subject, that is, the pulsation. For example, as illustrated in FIGS. 8 and 9, when a force is being applied to the support 20 in the direction indicated by the arrow P, the housing 10 can be displaced about an axis S in a direction indicated by an arrow DU or an arrow DL. The axis S may be a portion at which the second abutment portion 12 of the housing 10 contacts the wrist of the subject.

In the present embodiment, the sensor 50, which operates in association with the first abutment portion 11, is coupled to the support 20 through the elastic member 70. Thus, the sensor 50 is given a somewhat free range of motion because of the flexibility of the elastic member 70. The flexibility of the elastic member 70 further makes it difficult to hinder the movement of the sensor 50. The elastic member 70 having appropriate elasticity deforms in accordance with the pulsation in the target region of the subject. In the electronic device 1 according to the present embodiment, therefore, the sensor 50 can sensitively detect the pulsation in the target region of the subject. In addition, the electronic device 1 according to the present embodiment is displaced in accordance with the pulse wave, which can eliminate the congestion of the subject and eliminate the pain of the subject. In this manner, in the present embodiment, the elastic member 70 may be deformable in accordance with the pulsation in the target region of the subject. Further, the elastic member 70 may be elastically deformed to such an extent that the pulsation in the target region of the subject is detectable by the sensor 50.

As described above, the electronic device 1 according to an embodiment can function as a small and lightweight measurement device. The electronic device 1 according to an embodiment is not only excellent in portability but also capable of extremely easily measuring biological information of the subject. In addition, the electronic device 1 according to an embodiment can maintain a free-standing posture before measurement or the like. This allows the subject to easily position the target region when bringing the target region into abutment against the first abutment portion 11. Further, in the electronic device 1 according to an embodiment, the base 80 or the support 20 may be pressed downward during measurement. This eliminates the need for the subject to perform fine adjustment of the force pressing the base 80 or the support 20 during measurement. The electronic device 1 according to an embodiment can therefore provide relatively stable measurement of biological information of the subject. The electronic device 1 according to an embodiment can further measure the biological information alone, without cooperating with any other external device or the like. In this case, no other accessory such as a cable may be carried. The electronic device 1 according to an embodiment can therefore increase usability.

In one embodiment, the electronic device 1 may include a mechanism such as a stopper between the housing 10 and the support 20. In FIGS. 8 and 9, as an example, the housing 10 includes the protruding portion 14, and the support 20 includes the receiving portion 26. That is, the housing 10 includes, as part of the connection portion to be connected to the support 20, the protruding portion 14. The support 20 includes, as part of the opening into which the connection portion of the housing 10 is inserted, the receiving portion 26, which can receive the protruding portion 14. In the following, the protruding portion 14 and the receiving portion 26 are collectively referred to also as a “stopper (14, 26)”.

As illustrated in FIGS. 8 and 9, the stopper (14, 26) is formed only in a portion of the insertion portion of the housing 10 and the opening in the support 20 where the housing 10 and the support 20 are connected to each other. For example, in the example illustrated in FIGS. 8 and 9, the stopper (14, 26) is formed only at the lower end of a portion where the housing 10 and the support 20 are connected to each other. In contrast, the stopper (14, 26) is not formed at the upper end or the like of the portion where the housing 10 and the support 20 are connected to each other. In one embodiment, the stopper (14, 26) may not be formed at the upper end of the portion where the housing 10 and the support 20 are connected to each other or a portion other than the lower end of the portion where the housing 10 and the support 20 are connected to each other.

As described above, the stopper (14, 26) is provided only in a portion, which makes it difficult to suppress the movement of the housing 10 relative to the support 20 even when the subject relatively strongly presses the target region against the support 20. For example, in the situation illustrated in FIG. 8, the subject does not strongly press the target region against the support 20, and thus the protruding portion 14 and the receiving portion 26 do not abut against each other. In the situation illustrated in FIG. 9, in contrast, the subject strongly presses the target region against the support 20. Thus, the elastic member 70 is deformed, which causes a displacement of the housing 10 relative to the support 20. As a result, the protruding portion 14 and the receiving portion 26 abut against each other. Even in this case, the housing 10 and the support 20 do not abut against each other in a portion other than the portion where the protruding portion 14 and the receiving portion 26 abut against each other. Thus, the movement of the housing 10 relative to the support 20 in the manner indicated by the arrow DL illustrated in the drawing is somewhat suppressed, whereas the movement of the housing 10 relative to the support 20 in the manner indicated by the arrow UL illustrated in the drawing is not substantially suppressed. Thus, the movement of the housing 10 relative to the support 20 is less likely to be suppressed even when the subject relatively strongly presses the target region against the support 20.

In FIGS. 8 and 9, the housing 10 includes the protruding portion 14, and the support 20 includes the receiving portion 26. However, the housing 10 and the support 20 may have opposite configurations. That is, in one embodiment, the housing 10 may include the receiving portion 26, and the support 20 may include the protruding portion 14.

In this manner, the electronic device 1 according to an embodiment may include the stopper (14, 26). The stopper (14, 26) may include the protruding portion 14 and the receiving portion 26. The protruding portion 14 may be formed in one of the housing 10 and the support 20. The receiving portion 26 may be formed in the other of the housing 10 and the support 20. In the stopper (14, 26), the receiving portion 26 may be capable of receiving the protruding portion 14. In one embodiment, the stopper (14, 26) may allow the housing 10 to partially abut against the support 20 in response to the housing 10 being displaced with respect to the support 20 due to a deformation of the elastic member 70.

In the present embodiment, the sensor 50 may be, for example, a sensor that detects, for each of a plurality of axes, at least one selected from the group consisting of the angle (inclination), angular speed, and angular acceleration of an object, such as a gyro sensor (gyroscope). In this case, the sensor 50 can detect complex motion based on the pulsation in the target region of the subject as the respective parameters for the plurality of axes. Alternatively, the sensor 50 may be a six-axis sensor that is a combination of a three-axis gyro sensor and a three-axis acceleration sensor.

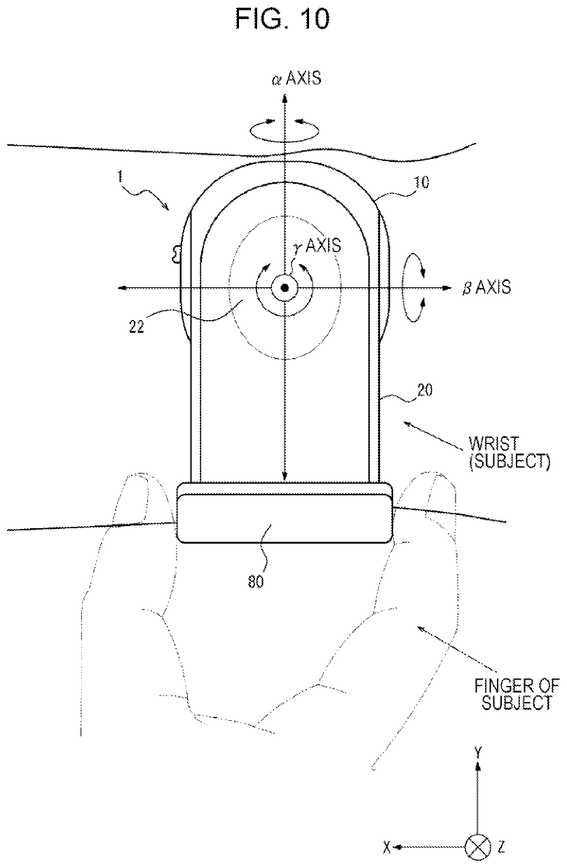

FIG. 10 is a diagram illustrating an example manner of using the electronic device 1. FIG. 10 is a diagram illustrating an enlarged view of the situation illustrated in FIG. 1 when viewed from a different viewpoint.

For example, as illustrated in FIG. 10, the sensor 50 built in the housing 10 of the electronic device 1 may detect a rotational movement about each of three axes, namely an a axis, a β axis, and a γ axis. The α axis may be, for example, an axis extending in a direction substantially orthogonal to the radial artery of the subject. The β axis may be, for example, an axis extending in a direction substantially parallel to the radial artery of the subject. The γ axis may be, for example, an axis extending in a direction substantially orthogonal to both the α axis and the β axis.

In the present embodiment, accordingly, the sensor 50 may detect pulsation in the target region of the subject as a portion of a rotational movement about a predetermined axis. Alternatively, the sensor 50 may detect pulsation in the target region of the subject as rotational movements on at least two axes or as rotational movements on three axes. In the present disclosure, the “rotational movement” may not necessarily be a movement including a displacement along a circular orbit by one or more turns. For example, in the present disclosure, the rotational movement may be, for example, a partial displacement along a circular orbit by less than one turn (for example, a displacement along an arc).

As illustrated in FIG. 10, the electronic device 1 according to the present embodiment can detect, for example, respective rotational movements about three axes by using the sensor 50. The electronic device 1 according to the present embodiment combines the plurality of results detected by the sensor 50 by, for example, adding them up, and can thus increase the detection sensitivity of the pulse wave of the subject. The computation, such as adding up, may be performed by the control unit 52, for example. In this case, the control unit 52 may calculate the index of the pulse wave based on the pulsation detected by the sensor 50.

For example, in the example illustrated in FIG. 10, the changes in signal strength with time based on the rotational movements of the sensor 50 about the α axis and the β axis have remarkable peaks based on the pulse wave of the subject. Thus, for example, the control unit 52 adds up the detection results for the α axis, the β axis, and the γ axis, and can thus increase the detection accuracy of the pulse wave of the subject. Accordingly, the electronic device 1 according to the present embodiment can improve the usefulness when the subject measures the pulse wave.

In one embodiment, the control unit 52 of the electronic device 1 may calculate the index of the pulse wave based on the pulsation detected by the sensor 50. In this case, the control unit 52 may combine (for example, add up) the results detected by the sensor 50 as rotational movements on at least two axes (for example, rotational movements on three axes). The electronic device 1 according to the present embodiment can detect pulse wave signals of a plurality of directions. Thus, the electronic device 1 according to the present embodiment combines detection results for a plurality of axes, thereby increasing the signal strength compared to a detection result for a single axis. Accordingly, the electronic device 1 according to the present embodiment can detect a signal having a good SN ratio and increase the detection sensitivity, achieving stable measurement.

In the detection result for the γ axis illustrated in FIG. 10, the peak based on the pulse wave of the subject is expected not to appear more noticeably than that in the detection result for the remaining α axis or β axis. In this manner, adding a detection result having a low signal level, such as the detection result for the γ axis, to a detection result for another axis may result in a reduction in SN ratio. In addition, a detection result having a low signal level may be mostly regarded as a noise component. In this case, the detection result having a low signal level may not contain a satisfactory pulse wave component. In the present embodiment, accordingly, if a detection result for an axis among the detection results for the plurality of axes is less than a predetermined threshold, the control unit 52 may not add the detection result for the axis.

For example, it is assumed that the pulsation of a certain subject is detected by the sensor 50 as the respective rotational movements about the α axis, the β axis, and the γ axis. As a result, the peak values in the detection results for the α axis, the axis, and the γ axis are each assumed to exceed a predetermined threshold. In this case, the control unit 52 may add up all of the detection result for the α axis, the detection result for the β axis, and the detection result for the γ axis to calculate the sum as the index of the pulse wave based on the pulsation detected by the sensor 50.

On the other hand, for example, as a result of detecting the pulsation of a certain subject, the peak values in the detection results for the α axis and the β axis are each assumed to exceed a predetermined threshold. However, the peak value in the detection result for the γ axis is assumed not to exceed a predetermined threshold. In this case, the control unit 52 may add up only the detection results for the α axis and the β axis to calculate the sum as the index of the pulse wave based on the pulsation detected by the sensor 50.

When performing such processing, the control unit 52 may set the thresholds, which are used as a reference to determine whether the detection results for the respective axes are to be added up, to be different or the same for the respective axes. In both cases, a threshold may be set appropriately so that the pulsation of the subject can be suitably detected in a detection result for each axis.

In this manner, in the electronic device 1 according to the present embodiment, the control unit 52 may combine only results having components equal to or greater than a predetermined threshold among the results detected by the sensor 50 as rotational movements on at least two axes. Thus, the electronic device 1 according to the present embodiment can suppress the reduction in the SN ratio of a detection result. Accordingly, the electronic device 1 according to the present embodiment can improve the usefulness when the subject measures the pulse wave.

As described above, when adding up detection results for a plurality of axes, merely adding up the detection results for the respective axes may cause a problem. This is presumably because the results detected by the sensor 50 do not match in polarity depending on the positional relationship between the direction of the pulsation of the subject and the sensor 50. For example, when the pulsation of the right hand of the subject is detected by using the sensor 50, the polarity of a detection result for a certain axis may be opposite to that when the pulsation of the left hand of the subject is detected by using the sensor 50.

For example, when the pulsation of the subject is detected, it is assumed that an upward peak is approximately periodically detected for a detection result for a certain axis. However, it is also assumed that a downward peak is approximately periodically detected for a detection result for another axis. In this manner, when detection results for a plurality of axes have opposite polarities, merely adding up the detection results may cause the peaks to be canceled out each other, and a satisfactory result may not be obtained.

In the present embodiment, accordingly, when detection results for a plurality of axes have opposite polarities, the control unit 52 may invert the polarity of the detection result for at least one axis before adding the detection result to the detection results for the other axes. For example, if detection results for two axes have opposite polarities, the control unit 52 may invert the polarity of the detection result for one axis in accordance with the other axis.

In this manner, in the electronic device 1 according to the present embodiment, the control unit 52 may combine the results detected by the sensor 50 as rotational movements on at least two axes after the polarities of the results are made to match each other. The electronic device 1 according to the present embodiment can increase the detection accuracy of the pulse wave of the subject. Accordingly, the electronic device 1 according to the present embodiment can improve the usefulness when the subject measures the pulse wave.

As described above, processing for matching the polarities of detection results for a plurality of axes by inverting the polarity of the detection result for at least one axis involves determining the directions of the polarities of the respective detection results. The directions of the polarities can be determined by using various methods. For example, the control unit 52 may deter mine whether the peak of the detection result for each axis is directed to the positive direction side or the negative direction side of the signal strength. Alternatively, for example, the control unit 52 may determine whether the peak of the detection result for each axis is larger or smaller than the average value of the signal. To invert the polarity of the detection result for at least one axis, the control unit 52 may multiply the detection result whose polarity is to be inverted by minus 1.

Further, after appropriately inverting the polarity of a detection result in the way described above, the control unit 52 may add or subtract a predetermined value to or from the entire detection result and then add the detection result to the detection results for the other axes. Alternatively, before adding up the detection results for the plurality of axes, the control unit 52 may appropriately weight the detection results for the respective axes or appropriately correct the detection results for the respective axes.

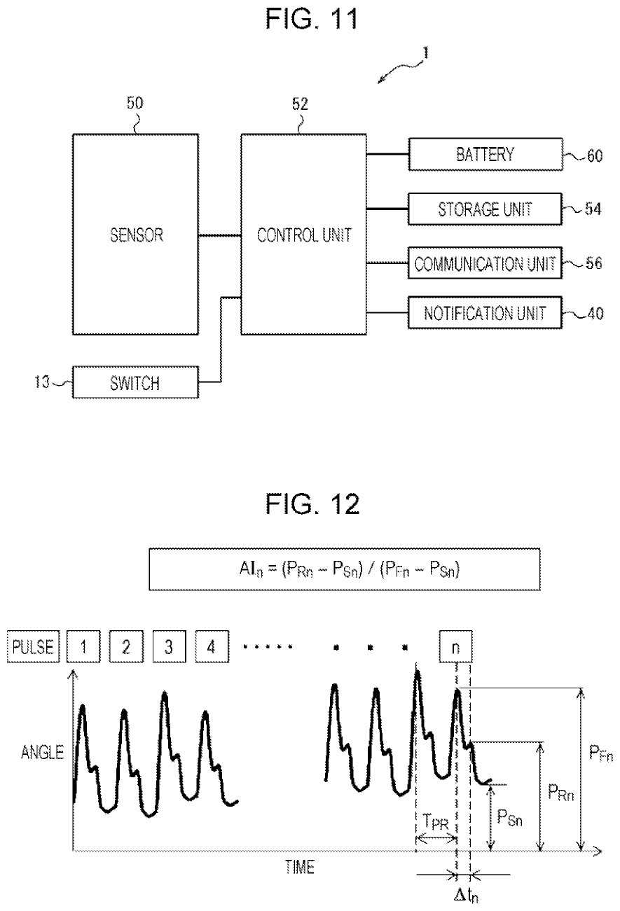

FIG. 11 is a functional block diagram illustrating a schematic configuration of the electronic device 1. The electronic device 1 illustrated in FIG. 11 includes the notification unit 40, the switch 13, the sensor 50, the control unit 52, the storage unit 54, the communication unit 56, and the battery 60. These functional units have been described above.

FIG. 12 is a diagram illustrating an example of a pulse wave acquired at the wrist by using the electronic device 1. FIG. 12 illustrates a case where an angular speed sensor is used as the sensor 50 that senses pulsation. In FIG. 12, an angular speed acquired by the angular speed sensor is integrated with respect to time. The horizontal axis represents time, and the vertical axis represents angle. The acquired pulse wave may contain noise caused by, for example, body movement of the subject and may thus be corrected by a filter that removes DC (Direct Current) components to extract only the pulsation components.

A method for calculating the index based on the pulse wave from the acquired pulse wave will be described with reference to FIG. 12. The propagation of the pulse wave is a phenomenon in which a heartbeat caused by blood pumped out of the heart is transmitted through the wall of an artery or the blood. The heartbeat caused by blood pumped out of the heart reaches the periphery of limbs as a forward traveling wave, and a portion thereof is reflected by a blood vessel branch portion, a blood-vessel-diameter changing portion, or the like and returns as a reflected wave. The index based on the pulse wave is, for example, the pulse wave velocity PWV of the forward traveling wave, the magnitude P_{R} of the reflected wave of the pulse wave, the time difference Δt between the forward traveling wave and reflected wave of the pulse wave, the AI (Augmentation Index), which is represented by the ratio of the magnitudes of the forward traveling wave and reflected wave of the pulse wave, or the like.

The pulse wave illustrated in FIG. 12 is a pulse wave with n pulses of a user, where n is an integer equal to or greater than 1. The pulse wave is a composite wave in which a forward traveling wave generated by the ejection of blood from the heart and a reflected wave generated from the blood vessel branch or the blood-vessel-diameter changing portion overlap each other. In FIG. 12, the magnitude of the peak of the pulse wave resulting from the forward traveling wave for each pulse is denoted by P_{Fn}, the magnitude of the peak of the pulse wave resulting from the reflected wave for each pulse is denoted by P_{Rn}, and the minimum value of the pulse wave of each pulse is denoted by P_{Sn}. In FIG. 12, the interval between the peaks of pulses is denoted by T_{PR}.

The index based on the pulse wave is obtained by quantifying information obtained from the pulse wave. For example, the PWV which is one index based on the pulse wave, is calculated based on the difference in propagation time between pulse waves measured at two target regions such as an upper arm and an ankle and the distance between the two target regions. Specifically, the PWV is calculated by acquiring pulse waves at two points along an artery (for example, an upper arm and an ankle) in synchronization with each other and dividing a distance difference (L) between the two points by a time difference (PTT) between the pulse waves at the two points. For example, as the magnitude P_{R} of the reflected wave, which is one index based on the pulse wave, the magnitude P_{Rn} of a peak of the pulse wave resulting from the reflected wave may be calculated, or P_{Rave} obtained by averaging the n magnitudes may be calculated. For example, as the time difference Δt between the forward traveling wave and reflected wave of the pulse wave, which is one index based on the pulse wave, a time difference Δtₙ in a predetermined pulse may be calculated, or Δtₐᵥₑ obtained by averaging the n time differences may be calculated. For example, the AI, which is one index based on the pulse wave, is obtained by dividing the magnitude of the reflected wave by the magnitude of the forward traveling wave, and is expressed by AIₙ=(P_{Rn}−P_{Sn})/(P_{Fn}−P_{Sn}). AIₙ is the AI for each pulse. The AI may be obtained by, for example, measuring a pulse wave for several seconds, calculating an average value AIₐᵥₑ of AIₙ (n is an integer of 1 to n) for the respective pulses, and setting the average value AIₐᵥₑ as an index based on the pulse wave.

The pulse wave velocity PWV, the magnitude P_{R} of the reflected wave, the time difference Δt between the forward traveling wave and the reflected wave, and the AI change depending on the stiffness of the blood vessel wall, and can thus be used to estimate the state of arteriosclerosis. For example, if the blood vessel wall is stiff, the pulse wave velocity PWV is large. For example, if the blood vessel wall is stiff, the magnitude P_{R} of the reflected wave is large. For example, if the blood vessel wall is stiff, the time difference Δt between the forward traveling wave and the reflected wave is small. For example, if the blood vessel wall is stiff, the AI is large. The electronic device 1 can, in addition to estimating the state of arteriosclerosis, estimate blood fluidity (viscosity) by using these indices based on the pulse wave. In particular, the electronic device 1 can estimate a change in blood fluidity from a change in the index based on the pulse wave acquired from the same target region of the same subject in a period during which the state of arteriosclerosis does not substantially change (for example, within several days). The blood fluidity represents a measure of the ease of blood flow. For example, if the blood fluidity is low, the pulse wave velocity PWV is small. For example, if the blood fluidity is low, the magnitude P_{R} of the reflected wave is small. For example, if the blood fluidity is the time difference Δt between the forward traveling wave and the reflected wave is large. For example, if the blood fluidity is low, the AI is small.

In the present embodiment, the electronic device 1 calculates the pulse wave velocity PWV, the magnitude P_{R} of the reflected wave, the time difference Δt between the forward traveling wave and the reflected wave, and the AI as example indices based on the pulse wave. However, the indices based on the pulse wave are not limited thereto. For example, the electronic device 1 may use the posterior systolic blood pressure as an index based on the pulse wave.

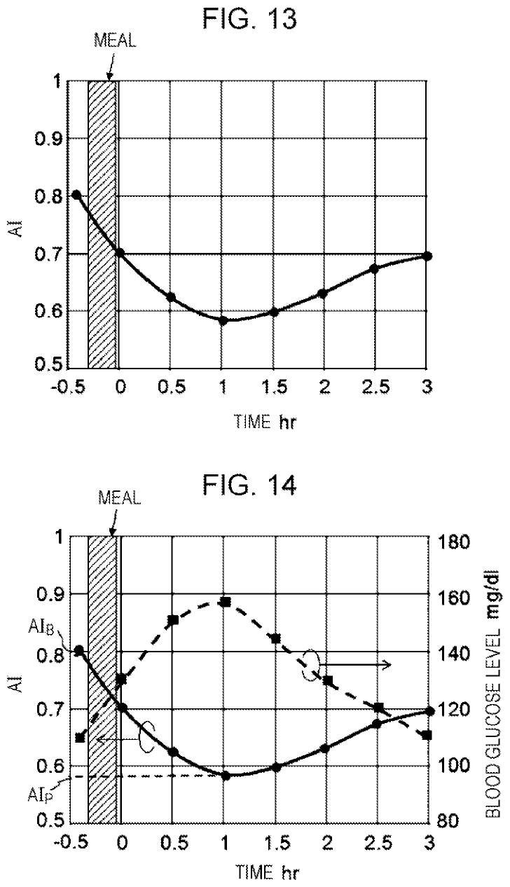

FIG. 13 is a diagram illustrating a time variation in calculated AI. In the present embodiment, the pulse wave was acquired for about 5 seconds by using the electronic device 1 including an angular speed sensor. The control unit 52 calculated the AI for each pulse from the acquired pulse wave and further calculated the average value AIₐᵥₑ of these AIs. In the present embodiment, the electronic device 1 acquired pulse waves at a plurality of timings before and after a meal, and calculated an average value of the AIs (hereinafter referred to as the AI) as an example index based on the acquired pulse waves. In FIG. 13, the horizontal axis represents the passage of time, with the first measurement time after the meal being 0. In FIG. 13, the vertical axis represents the AI calculated from the pulse wave acquired at that time. The pulse waves were acquired over the radial artery while the subject remained at rest.

The electronic device 1 acquired pulse waves before the meal, immediately after the meal, and every 30 minutes after the meal, and calculated a plurality of AIs on the basis of the respective pulse waves. The AI calculated from the pulse wave acquired before the meal was about 0.8. The AI immediately after the meal became smaller than that before the meal, and the AI reached the minimum extreme value at about 1 hour after the meal. The AI gradually increased until the measurement was finished at 3 hours after the meal.

The electronic device 1 can estimate a change in blood fluidity from change in calculated. AI. For example, if red blood cells, white blood cells, and platelets in blood are aggregated together or adhesion increases, blood fluidity decreases. For example, if the water content of plasma in blood becomes low, blood fluidity decreases. These changes in blood fluidity are caused by, for example, the glycolipid state described below or the health condition of the subject, such as heatstroke, dehydration, or hypothermia. Before the health condition of the subject becomes serious, the subject can recognize a change in their blood fluidity by using the electronic device 1 of the present embodiment. From the change in AI before and after the meal illustrated in FIG. 13, it can be estimated that the blood fluidity decreased after the meal, the blood fluidity decreased to the lowest level at about 1 hour after the meal, and then the blood fluidity gradually increased. The electronic device 1 may notify the subject of blood fluidity by expressing “thick” for a low blood fluidity state and “thin” for a high blood fluidity state. For example, the electronic device 1 may determine whether the blood is “thick” or “thin” on the basis of the average value of AIs for the age of the subject. The electronic device 1 may determine that the blood is “thin” if the calculated AI is larger than the average value, and determine that the blood is “thick” if the calculated AI is smaller than the average value. The electronic device 1 may determine whether the blood is “thick” or “thin” on the basis of, for example, the AI before the meal. The electronic device 1 may compare the AI after the meal with the AI before the meal and estimate the degree to which the blood is “thick”. The electronic device 1 can use, for example, the AI before the meal, that is, the AI during fasting, as an index for the vascular age (vascular stiffness) of the subject. For example, the electronic device 1 calculates an amount of change in calculated AI on the basis of the AI of the subject before the meal, that is, during fasting, thereby reducing estimation errors based on the vascular age (vascular stiffness) of the subject. As a result, a change in blood fluidity can be more accurately estimated.

FIG. 14 is a diagram illustrating a calculated AI and a measurement result of blood glucose level. The pulse wave acquisition method and the AI calculation method are the same as those in the embodiment illustrated in FIG. 13. In FIG. 14, the right vertical axis represents blood glucose level in blood, and the left vertical axis represents calculated AI. In FIG. 14, the solid line indicates an AI calculated from an acquired pulse wave, and the dotted line indicates a measured blood glucose level. The blood glucose level was measured immediately after the acquisition of the pulse wave. The blood glucose level was measured by using the blood glucose meter “Medisafe Fit”, manufactured by Terumo Corporation. Compared to the blood glucose level before the meal, the blood glucose level immediately after the meal increased by about 20 mg/dl. The blood glucose level reached the maximum extreme value at about 1 hour after the meal. Thereafter, the blood glucose level gradually decreased until the measurement was finished, and became almost the same as the blood glucose level before the meal at about 3 hours after the meal.

As illustrated in FIG. 14, the blood glucose levels before and after a meal have a negative correlation with the AI calculated from the pulse wave. As the blood glucose level increases, glucose in blood causes aggregation of red blood cells and platelets or increases adhesion, and, as a result, blood fluidity may decrease. A decrease in blood fluidity may decrease the pulse wave velocity PWV. A decrease in pulse wave velocity PWV may increase the time difference Δt between the forward traveling wave and the reflected wave. An increase in the time difference Δt between the forward traveling wave and the reflected wave may cause the magnitude P_{R} of the reflected wave to decrease compared to the magnitude P_{F} of the forward traveling wave. A decrease in the magnitude P_{R} of the reflected wave compared to the magnitude P_{F} of the forward traveling wave may decrease the AI. Since the AI within several hours (in the present embodiment, 3 hours) after the meal has a correlation with the blood glucose level, the variation in the blood glucose level of the subject can be estimated from the variation in AI. If the blood glucose level of the subject is measured in advance and the correlation with the AI is acquired, the electronic device 1 can estimate the blood glucose level of the subject from the calculated AI.

The electronic device 1 can estimate the state of glucose metabolism of the subject on the basis of the time of occurrence of Alp, which is the minimum extreme value of the AI detected for the first time after the meal. The electronic device 1 estimates, for example, the blood glucose level as the state of glucose metabolism. In an example estimation of the state of glucose metabolism, for example, if the minimum extreme value Alp of the AI detected for the first time after the meal is detected after a lapse of a predetermined time or longer (for example, about 1.5 hours or longer after the meal), the electronic device 1 can estimate that the subject has a glucose metabolism disorder (or is a patient with diabetes).

The electronic device 1 can estimate the state of glucose metabolism of the subject on the basis of the difference (AI_{B}−AI_{P}) between AI_{B}, which is the AI before the meal, and AI_{P}, which is the minimum extreme value of the AI detected for the first time after the meal. In an example estimation of the state of glucose metabolism, for example, if (AI_{B}−AI_{P}) is equal to or greater than a predetermined value (for example, 0.5 or more), the subject can be estimated to have a glucose metabolism disorder (or be a patient with postprandial hyperglycemia).

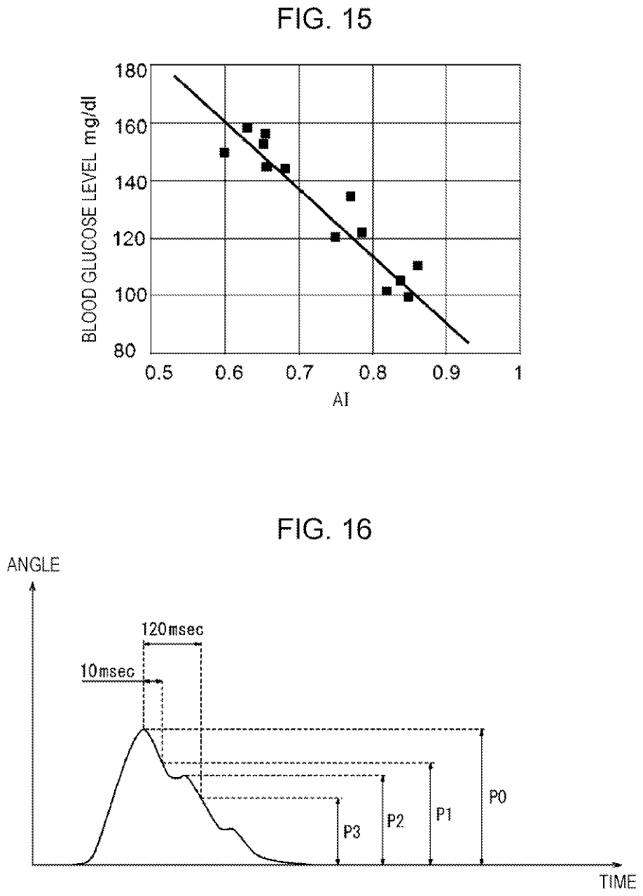

FIG. 15 is a diagram illustrating the relationship between the calculated. AI and the blood glucose level. The calculated AI and the blood glucose level were acquired within 1 hour after the meal, within which the blood glucose level varies greatly. The data in FIG. 15 includes a plurality of different pieces of data after the meal for the same subject. As illustrated in FIG. 15, the calculated AI and the blood glucose level exhibited a negative correlation. The correlation coefficient between the calculated AI and the blood glucose level was 0.9 or more and exhibited a very high correlation. For example, the correlation between the calculated AI and the blood glucose level illustrated in FIG. 15 may be acquired for each subject in advance, thus allowing the electronic device 1 to estimate the blood glucose level of the subject from the calculated AI.

Estimation of a blood pressure level (and a blood glucose level) of a subject by using the electronic device 1 according to an embodiment will be described.

A method for estimating a blood pressure level from a feature value of a pulse wave has already been proposed (for example, see Patent Literature 1 described above). The blood pressure of the subject varies after the subject has eaten a meal. Eating a meal relatively largely affects blood pressure variations. Accordingly, a method is desired for estimating the blood pressure level from the feature value of the pulse wave in consideration of the influence of the meal. An existing cuff-type sphygmomanometer measures the blood pressure level of the subject by using a cuff. In contrast, the electronic device 1 described above can also estimate the blood pressure level of the subject from the pulse wave of the subject detected without using a cuff (cuff-less type). A method for estimating the blood pressure level (and the blood glucose level) of the subject from the pulse wave of the subject detected without using a cuff by using the electronic device 1 described above in consideration of the influence of the meal will be described hereinafter.

The estimation of the blood pressure level (and the blood glucose level) of the subject by using the electronic device 1 can be mainly divided into the following two phases.

(1) Learning model generation phase: A phase of generating a learning model (estimation formula), which is used for estimation of a blood pressure level (and a blood glucose level) of a subject, by machine learning or the like by using AI (Artificial Intelligence), for example.

(2) Subject blood pressure level (and blood glucose level) estimation phase: A phase of estimating the blood pressure level (and the blood glucose level) of the subject on the basis of the pulse wave acquired by the electronic device 1, by using the learning model (estimation formula) generated in (1).

These phases will be described in more detail hereinafter.

First, in the learning model generation phase (1) described above, data indicating an index of a pulse wave of a person at a first time point and an index of a pulse wave of the person at a second time point may be collected to generate a learning model (estimation formula) that also takes into consideration the influence of the meal of the subject.

In this specification, the first time point may be a predetermined amount of time before a meal or a predetermined amount of time or less before a meal. The predetermined amount of time can be appropriately set. For example, the first time point may be 1 hour, 3 hours, 6 hours, or the like before a meal. Alternatively, for example, the first time point may be 1 hour or less, 3 hours or less, 6 hours or less, or the like before a meal. In this specification, the first time point may be a predetermined amount of time after the most recent meal, a predetermined amount of time or more after the most recent meal, or the like, for example. The predetermined amount of time can be appropriately set. For example, the first time point may be 1 hour, 3 hours, 6 hours, or the like after a meal. Alternatively, for example, the first time point may be 1 hour or more, 3 hours or more, 6 hours or more, or the like after a meal. In this specification, the first time point may be designated as the occurrence time of a specific activity such as a medical examination. In this specification, the first time point may be during fasting or may be a time point at which the subject recognizes the feeling of hunger, or the like, for example.

In this specification, the second time point may be after a meal. More specifically, in this specification, the second time point may be a predetermined amount of time after the most recent meal, a predetermined amount of time or more after the most recent meal, or the like. For example, the second time point may be 1 hour, 3 hours, 6 hours, or the like after a meal. Alternatively, for example, the second time point may be 1 hour or more, 3 hours or more, 6 hours or more, or the like after a meal.

In one embodiment, the first time point and/or the second time point is not limited to that in the examples described above and may be appropriately set by a user or the like, for example. As an example, collection of data indicating an index of a pulse wave of a person during fasting and an index of a pulse wave of the person after a meal will be described hereinafter. That is, in the learning model generation phase (1) described above, a meal tolerance test may be performed. The data to be collected in the learning model generation phase (1) described above may be based on the following three patterns.

First pattern: A measured value of a pulse wave before a meal, a measured value of a blood glucose level during fasting, and a measured value of a pulse wave after the meal

Second pattern: A measured value of a pulse wave before a meal, a measured value of a blood pressure level during fasting, and a measured value of a pulse wave after the meal

Third pattern: A measured value of a pulse wave before a meal and a measured value of a pulse wave after the meal

The patterns described above may be used in combination as appropriate. In addition, the pulse wave before or after a meal may be represented by a pulse rate or an augmentation index (A index) described below.

In the collection of the measured values described above, the pulse wave may be measured by the electronic device 1. In the collection of the measured values described above, the blood pressure level may be measured by any sphygmomanometer. In the collection of the measured values described above, the blood glucose level may be measured by any blood glucose meter, or known data such as measurement data in a medical examination may be used.

The applicant conducted a demonstration experiment for estimating a blood pressure level (and a blood glucose level) of a subject. The meal in a meal tolerance test was breakfast. That is, a person who underwent a meal tolerance test was subjected to measurement from 9:30 am to 12:30 pm without taking breakfast or medications. In this demonstration experiment, first, the pulse wave was measured by the electronic device 1, and then the blood glucose level was measured from a fingertip by the blood glucose meter. After the completion of the measurement, the person who underwent the meal tolerance test ingested a 623-kcal meal containing 86 g of carbohydrates, 18 g of lipids, and 30 g of proteins as a test meal, and the measurement of the same items was performed 1 hour later. Pulse wave measurement was performed on 60 subjects twice before the meal and twice after the meal, and blood glucose level measurement was performed on the 60 subjects once before the meal and once after the meal. Therefore, when the number of subjects was 60, the number of pieces of pulse wave data was 4×60=240, and the number of pieces of blood glucose level data was 2×60=120. The number of subjects may be any number other than 60.

The index of a pulse wave acquired (detected) by (the sensor 50 of) the electronic device 1 is defined. The applicant has focused on the fact that the shape of the pulse wave of a peripheral artery is affected by meals and that the augmentation index (A index), which is an index of wave reflections in a blood vessel, changes. The A index is an indicator representing the ratio of the magnitudes of the forward traveling wave and the reflected wave of the pulse wave. The electronic device 1 according to an embodiment may perform regression analysis of the A index and the blood glucose level. In this case, the regression analysis may be based on ensemble learning, which is an AI (Artificial Intelligence) learning method. Three A indices are defined in the following way as indices of a pulse wave acquired (detected) by the electronic device 1.

FIG. 16 is a diagram illustrating an example of a pulse wave detected by, for example, the electronic device 1, as in FIG. 12. FIG. 16 illustrates only one cycle of the pulse wave illustrated in FIG. 12. For example, in FIG. 16, the A index represented by P1/P0 is defined as a first augmentation index AI1. In FIG. 16, the A index represented by P2/P0 is defined as a second augmentation index AI2. In FIG. 16, further, the A index represented by P3/P0 is defined as a third augmentation index AI3. In FIG. 16, P0 indicates a value at a peak of a pulse wave. Further, P1 indicates a value after a lapse of 100 milliseconds from the peak of the pulse wave, that is, the peak time (the timing of the peak) of P0. Further, P2 indicates a value at a peak of a reflected wave of the pulse wave. Further, P3 indicates a value after a lapse of 120 milliseconds from the peak of the pulse wave, that is, the peak time (the timing of the peak) of P0.

In the learning model generation phase (1) described above, the electronic device 1 may acquire pulse waves of at least one or more persons during fasting and after a meal. For enhanced accuracy, the electronic device 1 may acquire pulse waves of a plurality of persons during fasting and after a meal. In the demonstration experiment conducted by the applicant, 60 persons underwent the meal tolerance test.

The electronic device 1 according to an embodiment may perform regression analysis based on machine learning in the learning model generation phase (1) described above. In the generation of a learning model to be used for estimation of the blood pressure level of the subject, that is, in the use of, for example, the blood pressure level as the objective variable, information on the subject, such as the age of the subject, data during fasting, and data after a meal, for example, may be used as explanatory variables. In one example, the information on the subject does not include the gender of the subject. The data during fasting may include the blood glucose level, the pulse rate, the first augmentation index AIL the second augmentation index AI2, and the third augmentation index AI3 of the subject. The data after a meal may include the pulse rate, the first augmentation index AIL the second augmentation index AI2, and the third augmentation index AI3 of the subject.

In the generation of a learning model to be used for estimation of the blood glucose level of the subject, that is, in the use of, for example, the blood glucose level as the objective variable, information on the subject, such as the age of the subject, data during fasting, and data after a meal, for example, may be used as explanatory variables. In one example, the information on the subject does not include the gender of the subject. The data during fasting may include the blood glucose level, the pulse rate, the first augmentation index AIL the second augmentation index AI2, and the third augmentation index AI3 of the subject. The data after a meal may include the pulse rate, the first augmentation index AIL the second augmentation index AI2, and the third augmentation index AI3 of the subject.

As described above, the electronic device 1 according to an embodiment can use the same explanatory variables to generate a learning model to be used for estimation of the blood pressure level of the subject and a learning model to be used for estimation of the blood glucose level of the subject. Accordingly, the electronic device 1 according to an embodiment can simultaneously estimate the blood pressure level and the blood glucose level of the subject by using such learning models.

The subject blood pressure level (and blood glucose level) estimation phase (2) described above will be described. The subject blood pressure level (and blood glucose level) estimation phase (2) is a phase of estimating the blood pressure level (and the blood glucose level) of the subject on the basis of the pulse wave acquired by the electronic device 1, by using the learning model (estimation formula) generated in (1).

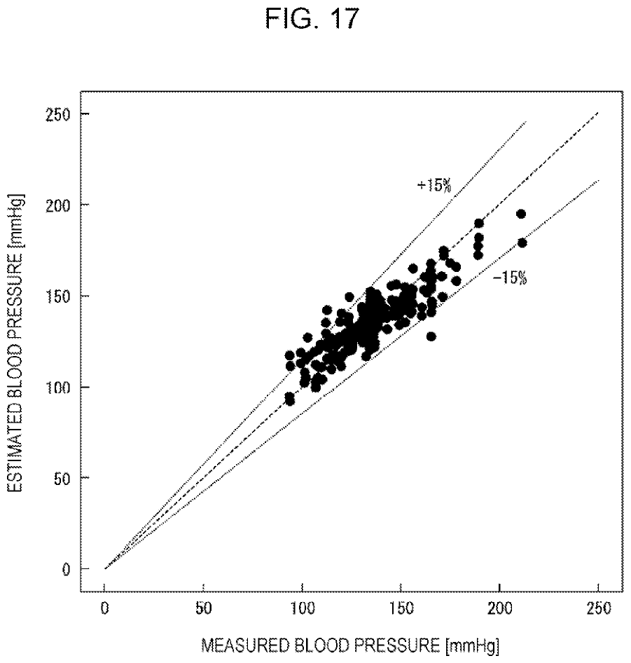

FIGS. 17 to 19 are diagrams illustrating results of regression analysis as an example of estimation of a blood pressure level by using the electronic device 1 according to an embodiment. In FIGS. 17 to 19, the horizontal axis represents a blood pressure level (systolic blood pressure) measured at an upper arm of a person, and the vertical axis represents an estimated blood pressure level.

FIG. 17 illustrates an example in which the electronic device 1 according to an embodiment estimates a blood pressure level by using a learning model generated based on data collected in the first pattern described above. That is, in FIG. 17, the explanatory variables include data of a blood glucose level during fasting. In the example illustrated in FIG. 17, the average of the correlation coefficients was calculated as 0.87, and the average of the coefficients of variation was calculated as 7.3.

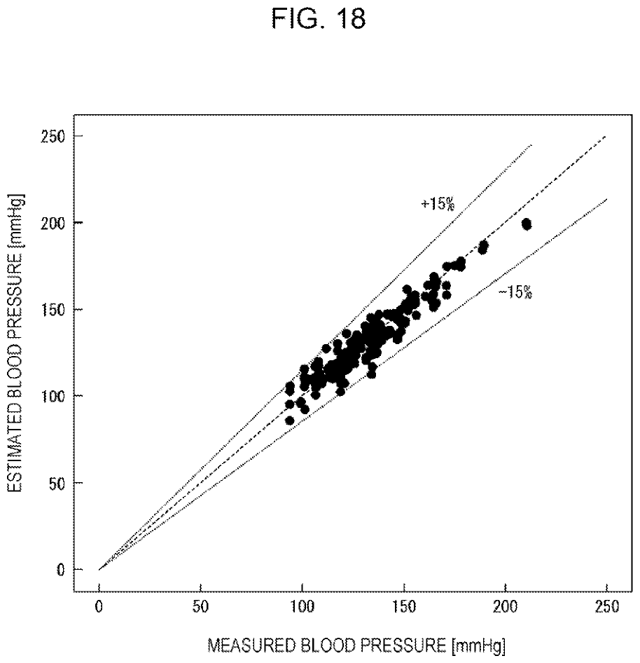

FIG. 18 illustrates an example in which the electronic device 1 according to an embodiment estimates a blood pressure level by using a learning model generated based on data collected in the second pattern described above. That is, in FIG. 18, the explanatory variables do not include data of a blood glucose level during fasting, but include data of a blood pressure level during fasting. In the example illustrated in FIG. 18, the average of the correlation coefficients was calculated as 0.95, and the average of the coefficients of variation was calculated as 4.5.

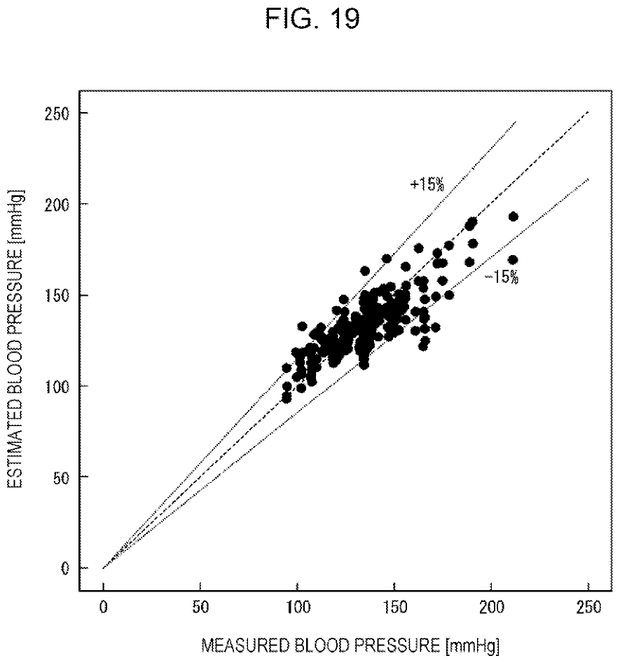

FIG. 19 illustrates an example in which the electronic device 1 according to an embodiment estimates a blood pressure level by using a learning model generated based on data collected in the third pattern described above. That is, in FIG. 19, the explanatory variables do not include data of a blood glucose level during fasting or data of a blood pressure level during fasting. In the example illustrated in FIG. 19, the average of the correlation coefficients was calculated as 0.80, and the average of the coefficients of variation was calculated as 8.9.

An existing cuff-less sphygmomanometer is typically unable to support a change in blood pressure due to the influence of a meal. In contrast, the electronic device 1 according to an embodiment generates a learning model (estimation formula) through a meal tolerance test and thus can address the influence of a meal.

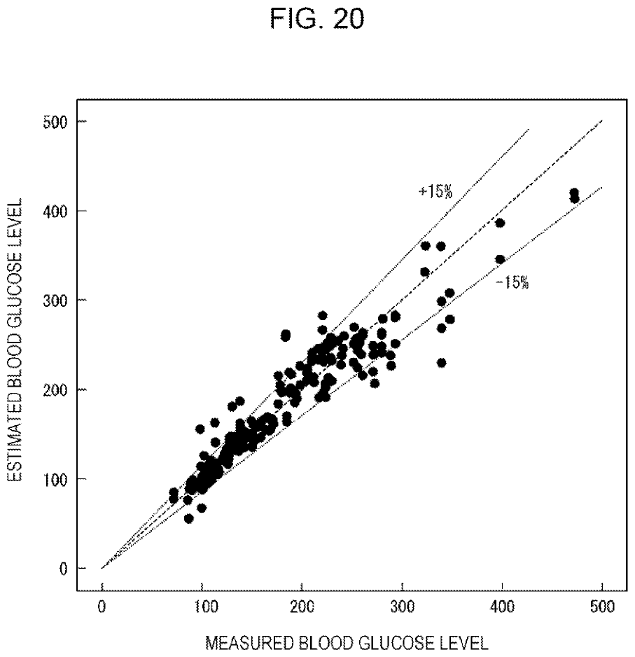

FIG. 20 is a diagram illustrating a result of regression analysis as an example of estimation of a blood glucose level by using the electronic device 1 according to an embodiment. In FIG. 20, the horizontal axis represents a blood glucose level measured from a fingertip of a person, and the vertical axis represents an estimated blood glucose level.

FIG. 20 illustrates an example in which the electronic device 1 according to an embodiment estimates a blood glucose level by using a learning model generated based on data collected in the first pattern described above. That is, in FIG. 20, the explanatory variables include data of a blood glucose level during fasting, as in FIG. 17. In the example illustrated in FIG. 20, the average of the correlation coefficients was calculated as 0.95, and the average of the coefficients of variation was calculated as 12.4. The example illustrated in FIG. 20 is based on data of 60 persons (55 of whom are diabetic). The example illustrated in FIG. 20 indicates that the estimation errors of 90% of the data are kept within ±15%.

The operation of the electronic device 1 according to an embodiment will be described.

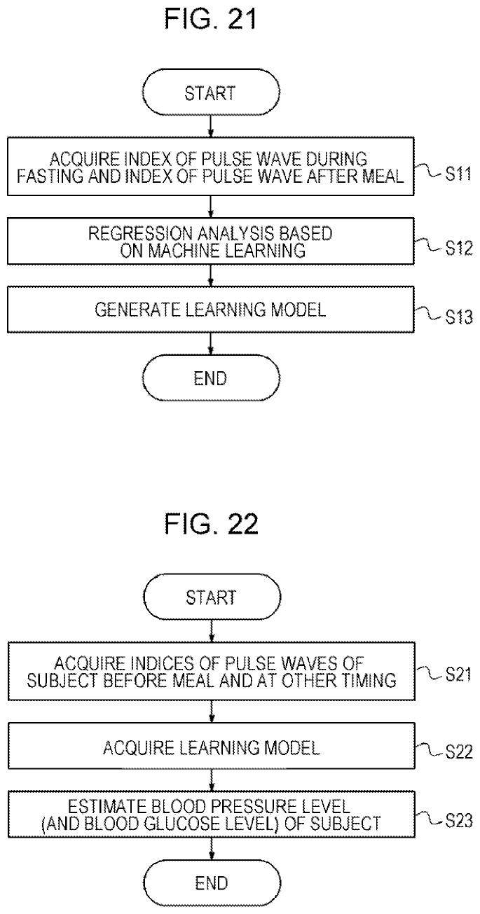

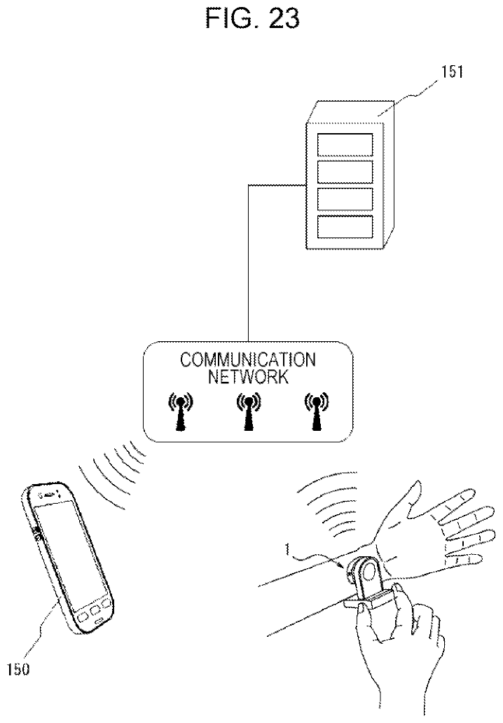

FIGS. 21 and 22 are flowcharts illustrating the operation of the electronic device 1 according to an embodiment. FIG. 21 is a flowchart illustrating the operation of the electronic device 1 in the learning model generation phase (1) described above. FIG. 22 is a flowchart illustrating the operation of the electronic device 1 in the subject blood pressure level (and blood glucose level) estimation phase (2) described above.

When the operation illustrated in FIG. 21 (the operation in the learning model generation phase) starts, the control unit 52 of the electronic device 1 acquires (collects) data of an index of a pulse wave during fasting and an index of a pulse wave after a meal (step S11). The time of fasting may typically refer to a time point before a meal, for example. The control unit 52 may store the data acquired in step S11 in, for example, the storage unit 54 or the like. In step S11, as described above, the control unit 52 may acquire pulse waves of at least one or more persons during fasting and after a meal. For enhanced accuracy, the electronic device 1 may acquire pulse waves of a plurality of persons during fasting and after a meal. For example, the electronic device 1 may perform a meal tolerance test on 60 persons as in the demonstration experiment (clinical test) conducted by the applicant. Alternatively, in step S11, the control unit 52 may select and acquire an appropriate explanatory variable from explanatory variables such as the pulse wave, blood glucose level, blood pressure level, and age of a person. In this case, the objective variable may be the blood pressure level of the person. As the population from which the data is collected, a group having a small bias may be appropriately selected within the specification of the electronic device 1 (for example, within the measurement range of the blood pressure). The data to be collected may also be based on a Gaussian distribution. The data to be collected may include data of the subject whose blood pressure level (and blood glucose level) is estimated in the subject blood pressure level (and blood glucose level) estimation phase (2) described above, or may include data of a person other than the subject.

After the index of the pulse wave during fasting and the index of the pulse wave after a meal are acquired (collected) in step S11, the control unit 52 performs regression analysis based on machine learning (step S12). In step S12, the control unit 52 may perform machine learning based on AI (Artificial Intelligence), for example. Alternatively, in step S12, for example, the control unit 52 may perform regression analysis based on machine learning after determining the objective variable and the explanatory variables. The objective variable may be, for example, the blood pressure level and/or the blood glucose level.

Techniques known as ensemble learning of machine learning include, for example, bagging, boosting, and stacking. In particular, regression analysis based on XGBoost is well known as a boosting technique. The electronic device 1 according to an embodiment may perform regression analysis based on XGBoost, for example. The electronic device 1 according to an embodiment may perform machine learning based on any other technique. Such various techniques of machine learning include known techniques, as appropriate. Thus, the technique of machine learning will not be described in more detail.

After regression analysis is performed in step S12, the control unit 52 generates a learning model (estimation formula) on the basis of the results of the regression analysis (step S13). The control unit 52 may store the learning model generated in step S13 in, for example, the storage unit 54 or the like.

As a result, the electronic device 1 according to an embodiment obtains a learning model based on machine learning. The obtained learning model may be a learning model output as a file even if, for example, the specific structure thereof is not clear.

As described above, the electronic device 1 according to an embodiment generates a learning model (estimation formula) to be used for estimation of a blood pressure level of a subject. In this case, the electronic device 1 according to an embodiment generates a learning model (estimation formula) indicating a relationship between a blood pressure level and a pulse wave associated with the blood pressure level, based on an index of a pulse wave of a person during fasting and an index of a pulse wave of the person after a meal.

Further, the electronic device 1 according to an embodiment generates a learning model (estimation formula) to be used for estimation of a blood pressure level and a blood glucose level of a subject. In this case, the electronic device 1 according to an embodiment generates a learning model (estimation formula) indicating a relationship between a blood pressure level and a pulse wave associated with the blood pressure level and a relationship between a blood glucose level and a pulse wave associated with the blood glucose level, based on an index of a pulse wave of a person during fasting and an index of a pulse wave of the person after a meal.

The index of the pulse wave may be an index indicating a ratio between a magnitude of a forward traveling wave of the pulse wave and a magnitude of a reflected wave of the pulse wave. The learning model may be generated further based on a blood pressure level of the person during fasting. The learning model may be generated further based on a blood pressure level of the person after a meal. The learning model may be generated further based on a blood glucose level of the person during fasting. The learning model may be generated further based on a blood glucose level of the person after a meal. Alternatively, the learning model may be generated in accordance with an elapsed amount of time after the person eats a meal. Alternatively, the learning model may be generated in accordance with whether the person is fasting or has eaten a meal.

Then, after the operation illustrated in FIG. 22 (the operation in the subject blood pressure level (and blood glucose level) estimation phase) starts, the control unit 52 of the electronic device 1 acquires (detects) the indices of the pulse waves of the subject before a meal and at another timing (step S21). The other timing in step S21 may be, for example, a time point after a meal or any other time point. In step S21, the electronic device 1 may acquire the indices of the pulse waves from the sensor 50. Further, in step S21, the control unit 52 may store the acquired indices of the pulse waves in, for example, the storage unit 54.

After the indices of the pulse waves are acquired in step S21, the control unit 52 acquires a learning model (step S22). The learning model acquired by the control unit 52 in step S22 may be the learning model generated in step S13 in FIG. 21. Alternatively, in step S22, the control unit 52 may read the learning model stored in the storage unit.

After the learning model is acquired or read in step S22, the control unit 52 estimates the blood pressure level (and the blood glucose level) of the subject by using the learning model (step S23). When using the learning model in step S23, the control unit 52 may use the explanatory variables of the subject corresponding to the explanatory variables in the learning model generation phase (1) described above. As a result, the electronic device 1 according to an embodiment estimates the blood pressure level (and the blood glucose level) of the subject with high accuracy.

As described above, the electronic device 1 according to an embodiment estimates a blood pressure level of a subject. In this case, the electronic device 1 according to an embodiment estimates the blood pressure level of the subject, based on indices of pulse waves acquired by the sensor 50, which include an index of a pulse wave of the subject before a meal and an index of a pulse wave of the subject at another timing. The electronic device 1 according to an embodiment estimates the blood pressure level of the subject by using a learning model that is generated based on an index of a pulse wave of a person during fasting and an index of a pulse wave of the person after a meal and that indicates a relationship between a blood pressure level and a pulse wave associated with the blood pressure level. Further, the electronic device 1 according to an embodiment estimates a blood pressure level and a blood glucose level of a subject. In this case, the electronic device 1 according to an embodiment estimates the blood pressure level and the blood glucose level of the subject, based on indices of pulse waves acquired by the sensor 50, which include an index of a pulse wave of the subject before a meal and an index of a pulse wave of the subject at another timing. The electronic device 1 according to an embodiment estimates the blood pressure level and the blood glucose level of the subject by using a learning model that is generated based on an index of a pulse wave of a person during fasting and an index of a pulse wave of the person after a meal and that indicates a relationship between a blood pressure level and a pulse wave associated with the blood pressure level and a relationship between a blood glucose level and a pulse wave associated with the blood glucose level.

In the related art, it is difficult to estimate a blood pressure level with high accuracy by using a feature value of a pulse wave. For example, a problem is that the estimated value of the blood pressure of the subject having a meal changes. Another problem is that since the feature value of the pulse wave greatly varies depending on an individual to be measured, the accuracy of estimation is not guaranteed if the same algorithm is applied to a relatively large number of persons. Still another problem is that due to the influence of other factors such as the tendency for a person with a high blood glucose level to have high blood pressure, the accuracy of estimation is not guaranteed if the blood pressure level is estimated by using only the feature value of the pulse wave.

In contrast, the electronic device 1 according to an embodiment takes into consideration the influence of meals, and the blood pressure level (and the blood glucose level) of even a person having a high blood glucose level can be estimated with high accuracy. Accordingly, the electronic device 1 according to an embodiment can estimate a blood pressure of a subject with high accuracy. Further, the electronic device 1 according to an embodiment can estimate a blood pressure level (and a blood glucose level) of a subject by using a cuff-less sensor. Accordingly, the electronic device 1 according to an embodiment can non-invasively estimate a blood pressure level (and a blood glucose level) of a subject.

FIG. 25 is a schematic diagram illustrating a schematic configuration of a system according to an embodiment. The system illustrated in FIG. 25 includes the electronic device 1, a server 151, a mobile terminal 150, and a communication network. As illustrated in FIG. 25, an index based on a pulse wave calculated by the electronic device 1 is transmitted to the server 151 via the communication network and is saved in the server 151 as personal information of the subject. The server 151 compares the index based on the pulse wave with previously acquired information of the subject and/or various databases to estimate the blood fluidity and the states of glucose metabolism and lipid metabolism of the subject. The server 151 further creates optimum advice for the subject. The server 151 returns the estimation results, the advice, and the like to the mobile terminal 150 possessed by the subject. The mobile terminal 150 notifies the subject of the received estimation results, advice, and the like through a display unit of the mobile terminal 150. Such a system can be constructed. Using the communication function of the electronic device 1 enables the server 151 to collect information from a plurality of users, resulting in a further increase in the accuracy of estimation. Since the mobile terminal 150 is used as a notification means, the electronic device 1 no longer requires the notification unit 40 and is further reduced in size. Further, since the server 151 estimates the blood fluidity and the states of glucose metabolism and lipid metabolism of the subject, the computational load on the control unit 52 of the electronic device 1 can be reduced. Further, since previously acquired information of the subject can be saved in the server 151, the load on the storage unit 54 of the electronic device 1 can be reduced. This results in further reduction in the size and complexity of the electronic device 1. In addition, the computational processing speed is also improved.

While the configuration of the system according to the present embodiment has been described in which the electronic device 1 and the mobile terminal 150 are connected to each other via the server 151 over the communication network, a system according to the present invention is not limited to this. The electronic device 1 and the mobile terminal 150 may be directly connected to each other over the communication network without using the server 151.

As described above, the electronic device 1 according to an embodiment may include the sensor 50 and the communication unit 56. The sensor 50 acquires a pulse wave of a subject. The communication unit 56 transmits, to another electronic device (for example, the server 151), information on pulse waves acquired by the sensor 50 or indices of the pulse waves, which are pulse waves of the subject before a meal and at another timing or indices of the pulse waves.

Characteristic examples have been described to fully and clearly disclose the present disclosure. However, the appended claims are not to be limited to the embodiments described above, but are to be configured to embody all variations and alternative configurations that may be created by a person skilled in the art in this technical field within the scope of the basic matter described herein.

For example, in the embodiments described above, the electronic device 1 includes an angular speed sensor as the sensor 50. However, the form of the electronic device 1 is not limited to this. The sensor 50 may include an optical pulse wave sensor including a light-emitting unit and a light-receiving unit, or may include a pressure sensor. In addition, the target region to be subjected to measurement of biological information by the electronic device 1 is not limited to the wrist of the subject. The sensor 50 may be placed over an artery, such as on a neck, an ankle, a thigh, or an ear.

For example, in the embodiments described above, the states of glucose metabolism and lipid metabolism of the subject are estimated on the basis of the first extreme value and the second extreme value of the index based on the pulse wave and the times thereof. However, the processing executed by the electronic device 1 is not limited to this. In some cases, only either extreme value may appear, or no extreme value may appear. The electronic device 1 may estimate the states of glucose metabolism and lipid metabolism of the subject on the basis of the overall tendency (for example, an integral value, Fourier transform, etc.) of the time variation in the index based on the calculated pulse wave. The electronic device 1 may estimate the states of glucose metabolism and lipid metabolism of the subject on the basis of a time range in which the index based on the pulse wave is equal to or less than a predetermined value, instead of by extracting extreme values of the index based on the pulse wave.

For example, in the embodiments described above, the blood fluidity before and after a meal is estimated. However, the processing executed by the electronic device 1 is not limited to this. The electronic device 1 may estimate the blood fluidity before and after exercise and during exercise, or may estimate the blood fluidity before and after bathing and during bathing.

In the embodiments described above, the electronic device 1 measures the pulse wave. However, the pulse wave may not necessarily be measured by using the electronic device 1. For example, the electronic device 1 may be connected to an information processing device such as a computer or a mobile phone in a wired or wireless manner, and angular speed information acquired by the sensor 50 may be transmitted to the information processing device. In this case, the information processing device may measure the pulse wave on the basis of the angular speed information. The information processing device may execute processing for estimating glucose metabolism and lipid metabolism, or the like. The information processing device connected to the electronic device 1 may execute various types of information processing. In this case, the electronic device 1 may not include the control unit 52, the storage unit 54, the notification unit 40, or the like. The electronic device 1 may be connected to the information processing device in a wired manner. In this case, the electronic device 1 may not include the battery 60 and may be supplied with electric power from the information processing device.

The control unit 52 of the electronic device 1 may estimate at least any one of glucose and lipid metabolism, blood glucose level, and lipid value from the index of the pulse wave. The electronic device 1 may function as a diet monitor that monitors the progress of a diet of the subject or a blood glucose meter that monitors the blood glucose level of the subject.

Aspects of the present disclosure will be presented as appendices hereinafter.

[Appendix 1]

An electronic device for generating a learning model to be used for estimation of a blood pressure level of a subject, wherein
the electronic device generates a learning model indicating a relationship between a blood pressure level and a pulse wave associated with the blood pressure level, based on an index of a pulse wave of a person during fasting and an index of a pulse wave of the person after a meal.

[Appendix 2]

An electronic device for estimating a blood pressure level of a subject by using a learning model generated based on an index of a pulse wave of a person during fasting and an index of a pulse wave of the person after a meal, the learning model indicating a relationship between a blood pressure level and a pulse wave associated with the blood pressure level,
estimation of the blood pressure level of the subject being based on an index of a pulse wave acquired by a sensor, the index including an index of a pulse wave of the subject before a meal and an index of a pulse wave of the subject at another timing.

[Appendix 3]

An electronic device for generating a learning model to be used for estimation of a blood pressure level and a blood glucose level of a subject, wherein
the electronic device generates a learning model indicating a relationship between a blood pressure level and a pulse wave associated with the blood pressure level and a relationship between a blood glucose level and a pulse wave associated with the blood glucose level, based on an index of a pulse wave of a person during fasting and an index of a pulse wave of the person after a meal.

[Appendix 4]

An electronic device for estimating a blood pressure level and a blood glucose level of a subject by using a learning model generated based on an index of a pulse wave of a person during fasting and an index of a pulse wave of the person after a meal, the learning model indicating a relationship between a blood pressure level and a pulse wave associated with the blood pressure level and a relationship between a blood glucose level and a pulse wave associated with the blood glucose level,
estimation of the blood pressure level of the subject being based on an index of a pulse wave acquired by a sensor, the index including an index of a pulse wave of the subject before a meal and an index of a pulse wave of the subject at another timing.

[Appendix 5]

The electronic device according to any one of appendices 1 to 4, wherein the learning model is generated further based on a blood pressure level of the person during fasting.

[Appendix 6]

The electronic device according to any one of appendices 1 to 5, wherein the learning model is generated further based on a blood pressure level of the person after a meal.

[Appendix 7]

The electronic device according to any one of appendices 1 to 6, wherein the learning model is generated further based on a blood glucose level of the person during fasting.

[Appendix 8]

The electronic device according to any one of appendices 1 to 7, wherein the learning model is generated further based on a blood glucose level of the person after a meal.

[Appendix 9]

The electronic device according to any one of appendices 1 to 8, wherein the learning model is generated in accordance with an elapsed amount of time after the person eats a meal.

[Appendix 10]

The electronic device according to any one of appendices 1 to 9, wherein the learning model is generated in accordance with whether the person is fasting or has eaten a meal.

[Appendix 11]

An electronic device including:
a sensor unit configured to acquire a pulse wave of a subject; and
a communication unit configured to transmit, to another electronic device, information on pulse waves of the subject before a meal and at another timing or on indices of the pulse waves, which are pulse waves acquired by the sensor unit or indices of the pulse waves.

[Appendix 12]

The electronic device according to any one of appendices 1 to 11, wherein the index of the pulse wave is an index indicating a ratio between a magnitude of a forward traveling wave of the pulse wave and a magnitude of a reflected wave of the pulse wave.

[Appendix 13]

A method for controlling an electronic device that generates a learning model to be used for estimation of a blood pressure level of a subject, the method including:
generating a learning model indicating a relationship between a blood pressure level and a pulse wave associated with the blood pressure level, based on an index of a pulse wave of a person during fasting and an index of a pulse wave of the person after a meal.

[Appendix 14]

A method for controlling an electronic device, the method including:
using a learning model generated based on an index of a pulse wave of a person during fasting and an index of a pulse wave of the person after a meal, the learning model indicating a relationship between a blood pressure level and a pulse wave associated with the blood pressure level; and
estimating a blood pressure level of a subject, based on an index of a pulse wave acquired by a sensor, the index including an index of a pulse wave of the subject before a meal and an index of a pulse wave of the subject at another timing.

[Appendix 15]

A method for controlling an electronic device that generates a learning model to be used for estimation of a blood pressure level and a blood glucose level of a subject, the method including:
generating a learning model indicating a relationship between a blood pressure level and a pulse wave associated with the blood pressure level and a relationship between a blood glucose level and a pulse wave associated with the blood glucose level, based on an index of a pulse wave of a person during fasting and an index of a pulse wave of the person after a meal.

[Appendix 16]

A method for controlling an electronic device, the method including:
using a learning model generated based on an index of a pulse wave of a person during fasting and an index of a pulse wave of the person after a meal, the learning model indicating a relationship between a blood pressure level and a pulse wave associated with the blood pressure level and a relationship between a blood glucose level and a pulse wave associated with the blood glucose level; and
estimating a blood pressure level and a blood glucose level of a subject, based on an index of a pulse wave acquired by a sensor, the index including an index of a pulse wave of the subject before a meal and an index of a pulse wave of the subject at another timing.

[Appendix 17]

A method for controlling an electronic device including a sensor unit that acquires a pulse wave of a subject, the method including:
transmitting, to another electronic device, information on pulse waves of the subject before a meal and at another timing or on indices of the pulse waves, which are pulse waves acquired by the sensor unit or indices of the pulse waves.

[Appendix 18]

A program for causing an electronic device that generates a learning model to be used for estimation of a blood pressure level of a subject, to perform:
generating a learning model indicating a relationship between a blood pressure level and a pulse wave associated with the blood pressure level, based on an index of a pulse wave of a person during fasting and an index of a pulse wave of the person after a meal.

[Appendix 19]

A program for causing an electronic device to perform:
using a learning model generated based on an index of a pulse wave of a person during fasting and an index of a pulse wave of the person after a meal, the learning model indicating a relationship between a blood pressure level and a pulse wave associated with the blood pressure level; and
estimating a blood pressure level of a subject, based on an index of a pulse wave acquired by a sensor, the index including an index of a pulse wave of the subject before a meal and an index of a pulse wave of the subject at another timing.

[Appendix 20]

A program for causing an electronic device that generates a learning model to be used for estimation of a blood pressure level and a blood glucose level of a subject, to perform:
generating a learning model indicating a relationship between a blood pressure level and a pulse wave associated with the blood pressure level and a relationship between a blood glucose level and a pulse wave associated with the blood glucose level, based on an index of a pulse wave of a person during fasting and an index of a pulse wave of the person after a meal.

[Appendix 21]

A program for causing an electronic device to perform:
using a learning model generated based on an index of a pulse wave of a person during fasting and an index of a pulse wave of the person after a meal, the learning model indicating a relationship between a blood pressure level and a pulse wave associated with the blood pressure level and a relationship between a blood glucose level and a pulse wave associated with the blood glucose level; and
estimating a blood pressure level and a blood glucose level of a subject, based on an index of a pulse wave acquired by a sensor, the index including an index of a pulse wave of the subject before a meal and an index of a pulse wave of the subject at another timing.

[Appendix 22]

A program for causing an electronic device including a sensor unit that acquires a pulse wave of a subject, to perform:
transmitting, to another electronic device, information on pulse waves of the subject before a meal and at another timing or on indices of the pulse waves, which are pulse waves acquired by the sensor unit or indices of the pulse waves.

### REFERENCE SIGNS

1 electronic device
10 housing
11 first abutment portion
12 second abutment portion
13 switch
14 protruding portion
20 support
22 rear surface portion
24 extension portion
26 receiving portion
30 substrate
40 notification unit
50 sensor
52 control unit
54 storage unit
56 communication unit
60 battery
70 elastic member
80 base
90 wrist rest portion
92 wrist abutment portion
150 mobile terminal
151 server

1. An electronic device for estimating a blood pressure level of a subject, comprising:
a controller configured to generate a learning model based on an index of a pulse wave of a person at a first time point and an index of a pulse wave of the person at a second time point later than the first time point, the learning model indicating a relationship between a blood pressure level and a pulse wave associated with the blood pressure level.

2. An electronic device for estimating a blood pressure level of a subject, comprising:
a controller configured to generate a learning model based on an index of a pulse wave of a person at a first time point and an index of a pulse wave of the person at a second time point later than the first time point, the learning model indicating a relationship between a blood pressure level and a pulse wave associated with the blood pressure level,
wherein the controller estimates the blood pressure level of the subject based on an index of a pulse wave acquired by a sensor, the index including an index of a pulse wave of the subject before a meal and an index of a pulse wave of the subject at another timing.

3. The electronic device according to claim 1, wherein the first time point is during fasting, and the second time point is after a meal.

4. The electronic device according to claim 1, wherein the learning model is generated further based on a blood pressure level at the first time point in addition to being based on the index of the pulse wave at the first time point and the index of the pulse wave at the second time point.

5. The electronic device according to claim 1, wherein the learning model is generated further based on a blood pressure level at the second time point in addition to being based on the index of the pulse wave at the first time point and the index of the pulse wave at the second time point.

6. The electronic device according to claim 1, wherein the learning model is generated further based on a blood glucose level at the first time point in addition to being based on the index of the pulse wave at the first time point and the index of the pulse wave at the second time point.

7. The electronic device according to claim 1, wherein the learning model is generated further based on a blood glucose level at the second time point in addition to being based on the index of the pulse wave at the first time point and the index of the pulse wave at the second time point.

8. The electronic device according to claim 3, wherein the learning model is generated in accordance with an elapsed amount of time after the person eats the meal.

9. The electronic device according to claim 3, wherein the learning model is generated in accordance with whether the person is fasting or has eaten the meal.

10. The electronic device according to claim 1, wherein the index of the pulse wave is an index indicating a ratio between a magnitude of a forward traveling wave of the pulse wave and a magnitude of a reflected wave of the pulse wave.

11. A method for controlling an electronic device, the method comprising:
generating a learning model based on an index of a pulse wave of a person at a first time point and an index of a pulse wave of the person at a second time point later than the first time point, the learning model indicating a relationship between a blood pressure level and a pulse wave associated with the blood pressure level; and
estimating a blood pressure level of a subject, based on an index of a pulse wave acquired by a sensor, the index including an index of a pulse wave of the subject before a meal and an index of a pulse wave of the subject at another timing.

12. (canceled)
